(19) United States
(12) Patent Application Publication
(10) Pub. No.: US 2024/0287074 A1
(43) Pub. Date: Aug. 29, 2024

(54) SALTS AND CRYSTALS

(71) Applicant: Kinoxis Therapeutics Pty Ltd, Camberwell (AU)

(72) Inventors: Hugh ALSOP, Camberwell (AU); Tristan REEKIE, Camberwell (AU); Ronnie Maxwell LAWRENCE, Camberwell (AU)

(21) Appl. No.: 18/044,190

(22) PCT Filed: Sep. 7, 2021
(86) PCT No.: PCT/AU2021/051033

§ 371 Date: Mar. 6, 2023

(30) Foreign Application Priority Data
Sep. 7, 2020 (AU) 2020903196

(51) Int. Cl. C07D 487/04; A61K 31/5517; A61P 25/36

(52) U.S. Cl. CPC C07D 487/04; A61K 31/5517; A61P 25/36

(57) ABSTRACT

This invention relates to solid forms of 1-methyl-1,4,5, IO-tetrahydropyrazolo[3,4-b][1,5]benzodiazepine freebase possessing desirable pharmacokinetic properties, such as low hygroscopicity and improved thermal stability. These solid forms include a crystalline form of 1-methyl-1,4,5,IO-tetrahydropyrazolo[3,4-b][1,5]benzodiazepine 5 freebase and phosphate and L-tartrate salts of 1-methyl-1,4,5,IO-tetrahydropyrazolo[3,4-b][1,5]benzodiazepine freebase. Pharmaceutical compositions, medicaments and kits comprising these solid forms are also described. Also provided are methods of using these forms to treat various diseases, conditions and disorders.

### CROSS-REFERENCE TO RELATED APPLICATION(S)

The present application claims priority from Australian Provisional Patent Application No. 2020903196 filed on 7 Sep. 2020, the entire contents of which is incorporated herein by reference.

### FIELD OF THE INVENTION

This invention relates to salts and crystals of 1-methyl-1,4,5,10-tetrahydropyrazolo[3,4-b][1,5]benzodiazepine.

### BACKGROUND OF THE INVENTION

1-Methyl-1,4,5,10-tetrahydropyrazolo[3,4-b][1,5]benzodiazepine dihydrochloride is described in WO2017/004674 as possessing biological activity similar to oxytocin agonists, without demonstrating significant binding affinity for the orthosteric oxytocin receptor binding sites or the orthosteric vasopressin receptor binding sites. As such, there is interest in developing pharmaceuticals comprising this compound.

1-Methyl-1,4,5,10-tetrahydropyrazolo[3,4-b][1,5]benzodiazepine has the following structure:

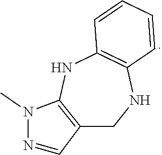

This compound may also be referred to as 1-methyl-1,4,5,10-tetrahydrobenzo[b]pyrazolo[3,4-e][1,4]diazepine. References to 1-methyl-1,4,5,10-tetrahydropyrazolo[3,4-b][1,5]benzodiazepine and 1-methyl-1,4,5,10-tetrahydrobenzo[b]pyrazolo[3,4-e][1,4]diazepine are intended to be interchangeable as used herein.

While able to induce promising biological activity, in further development of 1-methyl-1,4,5,10-tetrahydropyrazolo[3,4-b][1,5]benzodiazepine dihydrochloride it was discovered that this salt form demonstrated high hygroscopicity. Dynamic vapour sorption (DVS) analysis of the material indicated a form change above 60% relative humidity (RH) with the weight change reversible below 30% RH. While remaining a useful laboratory investigative tool, the high hygroscopicity makes further development of the dihydrochloride salt unsuitable.

There is therefore a need to provide alternative forms of 1-methyl-1,4,5,10-tetrahydropyrazolo[3,4-b][1,5]benzodiazepine for incorporation into pharmaceutical products. Advantageously, the alternative forms would provide material with lower hygroscopicity at 60% RH and above than the 1-methyl-1,4,5,10-tetrahydropyrazolo[3,4-b][1,5]benzodiazepine dihydrochloride salt.

All publications, patents and patent applications that may be cited herein are hereby incorporated by reference in their entirety.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The invention provides solid forms of 1-methyl-1,4,5,10-tetrahydropyrazolo[3,4-b][1,5]benzodiazepine (“compound of the invention”) possessing lower hygroscopicity than previously described pharmaceutically acceptable forms of the compound of the invention, including the dihydrochloride salt of the compound of the invention. The solid forms described herein may also demonstrate improved thermal stability compared to previously explored forms of the compounds and provide at least substantially equivalent biologically available compound of the invention to a subject following administration.

Surprisingly, the inventors have found that phosphoric acid and L-tartaric acid addition salts of the compound of the invention, as well as a crystalline form of the freebase of the compound of the invention possess one or more of these improved properties.

In one aspect, the invention provides a phosphoric acid addition salt of the compound of the invention. This phosphoric acid addition salt may alternatively be referred to as a phosphate salt of the compound of the invention.

In another aspect, the invention provides an L-tartaric acid addition salt of the compound of the invention. This L-tartaric acid addition salt may alternatively be referred to as an L-tartrate salt of the compound of the invention.

In some embodiments, the phosphate and/or L-tartrate salts of the compound of the invention are in a crystalline form.

In a further aspect, the invention provides a crystalline form of the compound of the invention. This crystalline form may also be referred to as a crystalline form of a freebase of the compound of the invention.

In another aspect, the invention provides a solid form, typically a crystalline form, of the compound of the invention selected from:
1-methyl-1,4,5,10-tetrahydropyrazolo[3,4-b][1,5]benzodiazepine freebase;
a phosphate salt of 1-methyl-1,4,5,10-tetrahydropyrazolo[3,4-b][1,5]benzodiazepine; or
an L-tartrate salt of 1-methyl-1,4,5,10-tetrahydropyrazolo[3,4-b][1,5]benzodiazepine.

Also described herein are methods of using these salt and crystalline forms of the compound of the invention, including in methods of:
treating or preventing antisocial behaviour in a subject, and/or
providing acute and long-term regulation of social behaviour in a subject, and/or
treating or preventing a substance abuse disorder in a subject, and/or
treating or preventing a social dysfunction in a subject, and/or
treating or preventing a psychiatric disorder in a subject as part of a therapy for a psychiatric disorder that features social dysfunction as a primary or secondary feature; and/or
causing weight loss in a subject; and/or
managing weight in a subject; and/or
suppressing an appetite for food in a subject; and/or
reducing the consumption of food by a subject; and/or
treating or preventing opioid withdrawal and/or a symptom associated with the opioid withdrawal in a subject.

As used herein, except where the context requires otherwise, the term “comprise” and variations of the term, such as “comprising”, “comprises” and “comprised”, are not intended to exclude further additives, components, integers or steps.

It must be noted that as used herein and in the appended claims, the singular forms “a”, “an” and “the” include plural reference unless the context clearly dictates otherwise. Thus, for example, a reference to “a symptom” and/or “at least one symptom” may include one or more symptoms, and so forth.

The term “and/or” can mean “and” or “or”.

The term “(s)” following a noun contemplates the singular or plural form, or both.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, 5%, ±1% or ±0.1% of that value.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1 shows an X-ray diffraction (XRD) pattern of crystalline 1-methyl-1,4,5,10-tetrahydropyrazolo[3,4-b][1,5]benzodiazepine freebase

FIG. 2 shows a thermogravimetric (TG) plot of an analysis of crystalline 1-methyl-1,4,5,10-tetrahydropyrazolo[3,4-b][1,5]benzodiazepine freebase

FIG. 3 shows a differential scanning calorimetry (DSC) plot of crystalline 1-methyl-1,4,5,10-tetrahydropyrazolo[3,4-b][1,5]benzodiazepine freebase

FIG. 4 shows a DVS isotherm plot of crystalline 1-methyl-1,4,5,10-tetrahydropyrazolo[3,4-b][1,5]benzodiazepine freebase

FIG. 5 shows a DVS change in mass plot of crystalline 1-methyl-1,4,5,10-tetrahydropyrazolo[3,4-b][1,5]benzodiazepine freebase

FIG. 6 shows a comparison of reference (upper) X-ray powder diffraction (XRPD) pattern of crystalline 1-methyl-1,4,5,10-tetrahydropyrazolo[3,4-b][1,5]benzodiazepine freebase with XRPD pattern after 1-week stability study described in examples 1 and 6

FIG. 7 shows an XRD pattern of a polymorphic form (phosphate form 1) of a phosphoric acid addition salt of 1-methyl-1,4,5,10-tetrahydropyrazolo[3,4-b][1,5]benzodiazepine

FIG. 8 shows a TGA/DTA plot of a polymorphic form (phosphate form 1) of a phosphoric acid addition salt of 1-methyl-1,4,5,10-tetrahydropyrazolo[3,4-b][1,5]benzodiazepine

FIG. 9 shows stacked XRD patterns of freebase (upper), and polymorphic form 1 (middle) and pattern 2 (lower) of the phosphoric acid addition salt of 1-methyl-1,4,5,10-tetrahydropyrazolo[3,4-b][1,5]benzodiazepine

FIG. 10 shows an XRD pattern of a crystalline form of an L-tartaric acid addition salt of 1-methyl-1,4,5,10-tetrahydropyrazolo[3,4-b][1,5]benzodiazepine

FIG. 11 shows TGA/DTA plots of a crystalline form of an L-tartaric acid addition salt of 1-methyl-1,4,5,10-tetrahydropyrazolo[3,4-b][1,5]benzodiazepine

FIG. 12 shows a DSC thermogram (first heat) of a crystalline form of an L-tartaric acid addition salt of 1-methyl-1,4,5,10-tetrahydropyrazolo[3,4-b][1,5]benzodiazepine

FIG. 13 shows a DVS isotherm plot of a crystalline form of an L-tartaric acid addition salt of 1-methyl-1,4,5,10-tetrahydropyrazolo[3,4-b][1,5]benzodiazepine

FIG. 14 shows a DVS change in mass plot of a crystalline form of an L-tartaric acid addition salt of 1-methyl-1,4,5,10-tetrahydropyrazolo[3,4-b][1,5]benzodiazepine

FIG. 15 shows a chart of mean plasma concentrations of compound of the invention in male Sprague Dawley rats following oral administration of the compound of invention (CMPD1) as a dihydrochloride salt (di-HCL) or as phosphate form 1 (phosphate salt) in saline based and methocel formulations at a target dose of 5 mg/kg. All doses and concentrations are expressed as the freebase equivalents. Data represent the mean±SD (n=3 animals/group).

FIG. 16 shows a chart of frequency of jumping by treatment group (vehicle only, oxycodone, oxycodone followed by phosphate form 1 and oxycodone followed by a dihydrochloride salt of the compound of the invention after oxycodone withdrawal was precipitated in C57BL/6 mice by naloxone administration (paw tremor results of Example 10).

FIG. 17 shows Gravimetric Vapour Sorption (GVS) isotherm plot of a phosphoric acid addition salt of 1-methyl-1,4,5,10-tetrahydropyrazolo[3,4-b][1,5]benzodiazepine (phosphate form 1).

FIG. 18 shows a GVS kinetic plot of a phosphoric acid addition salt of 1-methyl-1,4,5,10-tetrahydropyrazolo[3,4-b][1,5]benzodiazepine (phosphate form 1).

### DETAILED DESCRIPTION OF THE EMBODIMENTS

The invention relates to salt and/or crystalline forms of the compound of the invention. These salt and/or crystalline forms include:
a phosphoric acid addition salt of the compound of the invention;
an L-tartaric acid addition salt of the compound of the invention;
a crystalline form of the compound of the invention (freebase).

Collectively, the above salt and crystalline forms of the compound of the invention are referred to herein as the salts and/or crystals of the invention.

Each of the salts and/or crystals of the invention were surprisingly found to have desirable properties in terms of their low hygroscopicity while retaining bioavailability of the compound of the invention. The salt and/or crystalline forms described herein were the only forms of the compound of the invention possessing these properties from a screen of 18 acid counterions and 5 solvent systems (see Example 1).

The salts and/or crystals of the invention may be substantially non-hygroscopic when exposed to an environment with a minimum relative humidity of at least about 60% RH, 70% RH, 75% RH or 80% RH. The salts and/or crystals of the invention may be substantially non-hygroscopic when exposed to environments at a maximum relative humidity of not more than about 90%, 85%, 80% or 75%. The salts and/or crystals of the invention may be substantially non-hygroscopic when exposed to an environment having a relative humidity from any of these minimum values to any of these maximum values provided the minimum value is less than the maximum value. For example, in some embodiments, the salts and/or crystals of the invention are substantially non-hygroscopic when exposed to an environment at a relative humidity from about 60% to about 90% or from about 75% to about 85%. At relative humidities of about 90% RH and above the salts and/or crystals of the invention may increase in mass by no more than about 2 wt %, 1.5 wt %, 1 wt %, 0.9 wt %, 0.8 wt %, 0.7 wt %, 0.6 wt % or 0.5 wt % due to the absorption of water. The increase in mass may be measured by DVS, for example, according to any procedure described herein.

The salts and/or crystals of the invention may also be substantially stable for an extended period of time. For example, the salts and/or crystals may be stable for a period of 1 week, 1, 2, 3, 4, 5, 6 months or longer upon storage at 25° C. and 60% RH. The salts and/or crystals may also be stable for 1 week, 1, 2, 3, 4, 5, 6 months or longer upon storage under accelerated storage conditions, for example, at 40° C. at 75% RH. In some embodiments, the salts and/or crystals retain at least about 95%, 96%, 97%, 98%, 98.5% or 99% purity upon storage under any of these storage conditions.

The salts and/or crystals of the invention may be prepared from 1-methyl-1,4,5,10-tetrahydropyrazolo[3,4-b][1,5]benzodiazepine prepared by any suitable means. The synthesis of 1-methyl-1,4,5,10-tetrahydropyrazolo[3,4-b][1, 5]benzodiazepine and its dihydrochloride salt have been previously described, including in WO2017/004674 (U.S. Ser. No. 11/033,555) which is incorporated herein entirely by reference.

In some embodiments, the salts and/or crystals of the invention are prepared from 1-methyl-1,4,5,10-tetrahydropyrazolo[3,4-b][1,5]benzodiazepine prepared by reacting N-(1-methyl-1H-pyrazol-5-yl)-benzene-1,2-diamine with formaldehyde in a solvent and in the presence of an acid to provide 1-methyl-1,4,5,10-tetrahydropyrazolo[3,4-b][1,5]benzodiazepine. Any acid capable of mediating the reaction may be used. Suitable acids include acetic acid, phosphoric acid, and so on. As will be discussed further below, when phosphoric acid is included in this reaction step, the product may be the phosphoric acid addition salt of the compound of the invention.

In some embodiments, the salts and/or crystals of the invention are prepared from 1-methyl-1,4,5,10-tetrahydropyrazolo[3,4-b][1,5]benzodiazepine prepared by the following steps:
reacting 1-methyl-1H-pyrazol-5-amine and 1-fluoro-2-nitrobenzene in an organic solvent (typically tetrahydrofuran) in the presense of base (typically an alkoxide, such as potassium tert-butoxide) to provide 2-(1-methyl-1H-pyrazol-5-amino)-1-nitrobenzene;
reducing 2-(1-methyl-1H-pyrazol-5-amino)-1-nitrobenzene, typically in the presence of a palladium catalyst (such as palladium on carbon) in a polar solvent (eg a polar aprotic solvent such as ethyl acetate, acetonitrile and the like, or a protic solvent such as methanol and the like), to provide N-(1-methyl-1H-pyrazol-5-yl)-benzene-1,2-diamine; and
reacting N-(1-methyl-1H-pyrazol-5-yl)-benzene-1,2-diamine with formaldehyde in a solvent and in the presence of an acid to provide 1-methyl-1,4,5,10-tetrahydropyrazolo[3,4-b][1,5]benzodiazepine.

### Phosphoric Acid Addition Salt of 1-methyl-1,4,5,10-tetrahydropyrazolo[3,4-b][1,5]benzodiazepine

In a first aspect, the invention provides a phosphoric acid addition salt of 1-methyl-1,4,5,10-tetrahydropyrazolo[3,4-b][1,5]benzodiazepine. This salt form may be referred to herein as the phosphate salt of the invention.

The phosphate salt of the invention may be a hydrogen phosphate, dihydrogen phosphate or a phosphate salt of the compound of the invention. In some embodiments, the phosphate salt is a dihydrogen phosphate salt of the compound of the invention.

Typically, the phosphate salt of the invention is crystalline. It has been found that crystalline forms of the phosphate salt of the invention demonstrate polymorphism, with 2 distinct polymorphs identified, referred to herein as phosphate form 1 and phosphate pattern 2. It has also been found that phosphate form 1 is a stable crystalline form of the phosphate salt of the invention, while form 2 is unstable and converts to form 1 over time. Therefore, in some embodiments, the phosphate salt of the invention is provided as phosphate form 1.

Phosphate form 1 may be characterised by its X-ray diffraction (XRD) pattern. The XRD pattern for phosphate form 1 comprises characterising strong peaks at about 12° and about 18° 2θ.

Additionally, phosphate form 1 may be characterised by peaks in the XRD pattern at about 12°, 14°, 17.5°, 18°, 19°, 20°, 20.8°, 22.5°, 24°, 24.8°, 26°, 26.5° and 27.8° 2θ.

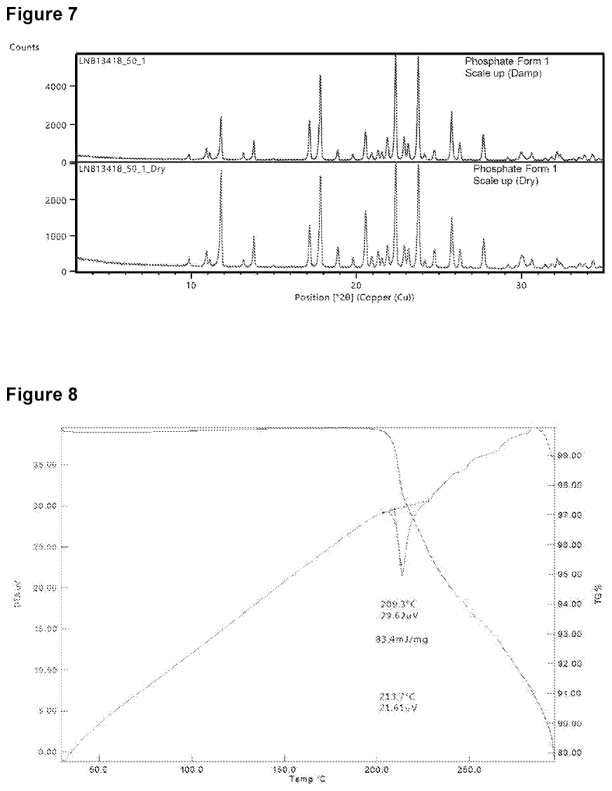

Typically, phosphate form 1 may be characterised by the XRD pattern shown in FIG. 7.

Phosphate form 1 may additionally or alternatively be characterised by its melting point. It has been found that the melting point of phosphate form 1 (about 200° C.) is higher by about 8° C. than the melting point for phosphate pattern 2 indicating that it has a higher degree of crystallinity and hence greater stability. DT analysis of phosphate pattern 2 showed a broad melting endotherm from an onset of about 201° C., with a peak at 206° C., which was followed immediately by thermal degradation. In contrast, DT analysis showed a large endothermic melting transition for phosphate form 1 from an onset of about 209° C. with a peak at 214° C. Therefore, in some embodiments the phosphate salt of the compound of the invention may have a melting point of about 200° C.

The phosphate salt of the compound of the invention is typically an anhydrous crystal. The anhydrous nature of this crystal form may be determined by thermogravimetric analysis.

The phosphate salt of the invention may remain substantially anhydrous and at purity levels that are substantially unchanged when stored under ambient conditions or under accelerated storage conditions. The accelerated storage conditions may comprise elevated temperature (eg 40° C. or 80° C.) and/or increased relative humidity. In some embodiments, the phosphate salt may remain substantially anhydrous upon storage for example for at least 1, 2 or 3 week(s), 1, 2, 3, 4, 5, 6 months or longer, at elevated temperature (eg 40° C.) and at up to about 60% RH, 70% RH or 75% RH. Typically, the phosphate salt will also remain stable under these storage conditions, remaining substantially pure throughout, for example resulting in up to about 2%, 1.5% or 1% degradation products detectable by HPLC. The HPLC may be carried out by any of the techniques described herein.

The phosphate salt of the invention may be prepared by any suitable means. The process may involve combination of phosphoric acid with the compound of the invention in a suitable solvent. This process may be carried out on isolated freebase material, or may be conducted in a 1-pot process with the final synthetic step of preparing the compound of the invention, where the salt is formed without isolating the freebase.

Extensive solvent screening was carried out to determine what conditions influenced formation of crystalline forms—form 1 and pattern 2 (see example 4). It was found that form 1 is the form that is provided under most conditions except in highly polar solvents such as water, N-methyl pyrrolidine (NMP) and dimethylsulfoxide (DMSO) due to the salt's solubility, when left to stand in concentrated solutions of ethyl acetate, methylisobutyl ketone and tert-butylmethylether having been subjected to heating cooling cycles, or when allowed to mature in tert-butylmethylether. Mixtures of form 1 and pattern 2 were obtained when ethyl formate (maturation and standing following heat cycling), isopropyl acetate (standing following heat cycling), methylethyl ketone (standing following heat cycling), and chloroform (trace pattern 2 on standing following heat cycling).

Accordingly, also provided is a process for preparing the phosphate salt of the invention, comprising
preparing a solution comprising 1-methyl-1,4,5,10-tetrahydropyrazolo[3,4-b][1,5]benzodiazepine freebase, a solvent, and phosphoric acid; and
separating excess solvent and phosphoric acid to provide the phosphate salt.

In some embodiment, the method further comprises preparing a crystallisation solution of the phosphate salt and a minimum volume of a crystallisation solvent to form the crystallisation solution. The crystallisation solution may be allowed to stand under ambient conditions and/or cooled and/or concentrated to allow crystal formation.

In embodiments where form 1 is desired, the crystallisation solvent typically does not comprise ethyl acetate, methylisobutyl ketone, tert-butylmethyl ether, ethyl formate, isopropyl acetate, and methyl ethyl ketone or a combination thereof. In some embodiments, the solvent further does not comprise chloroform.

When form 1 is desired, the crystallisation solvent may be selected from 1,4-dioxane, 2-butanol, 2-ethoxyethanol, 2-methyl tetrahydrofuran, 2-propanol, acetone, acetonitrile, methanol, anisole, ethanol, tetrahydrofuran, ethyleneglycol and water or a combination thereof.

When phosphate pattern 2 is desired, the crystallisation solvent is preferably tert-butylmethylether.

Also provided is a process for preparing the phosphate salt of the invention, comprising reacting N-(1-methyl-1H-pyrazol-5-yl)-benzene-1,2-diamine with formaldehyde in the presence of phosphoric acid in a solvent to provide the phosphoric acid addition salt of the compound of the invention.

The reaction of N-(1-methyl-1H-pyrazol-5-yl)-benzene-1,2-diamine with formaldehyde may be carried out in any suitable solvent, such as any of the crystallisation solvents described herein. The reaction of N-(1-methyl-1H-pyrazol-5-yl)-benzene-1,2-diamine with formaldehyde may further comprise forming a solution of N-(1-methyl-1H-pyrazol-5-yl)-benzene-1,2-diamine and phosphoric acid in a solvent, and adding formaldehyde to the solution. The solution may comprise any suitable solvent. In some embodiments, the solvent is an aqueous solvent. In some embodiments, the solvent is selected from 1,4-dioxane, 2-butanol, 2-ethoxyethanol, 2-methyl tetrahydrofuran, 2-propanol, acetone, acetonitrile, methanol, anisole, ethanol, tetrahydrofuran, ethyleneglycol and water or a combination thereof. In some embodiments, the solvent is selected from acetonitrile, water or a combination thereof. Combinations of solvents may comprise any suitable mixture of components, for example a 2 solvent mixture such as acetonitrile and water may be in a ratio by weight of from about 1:1 to about 2:1 acetonitrile to water.

The reaction of N-(1-methyl-1H-pyrazol-5-yl)-benzene-1,2-diamine with formaldehyde may progress at elevated temperatures. In some embodiments, the temperature of the reaction is from about 25° C. to about 50° C., about 25° C. to about 45° C. or about 35° C. to about 45° C. In some embodiments, the temperature of this reaction may be carried out at a temperature of about 30, 31, 32, 33, 34, 35, 36, 37, 38, 39, 40, 41, 42, 43, 44 or 45° C.

The reaction temperature may be from any of these temperatures to any other of these temperatures.

The reaction of N-(1-methyl-1H-pyrazol-5-yl)-benzene-1,2-diamine with formaldehyde may include any suitable amount of phosphoric acid. Typically, the phosphoric acid is present in this step in an amount of about 1 molar equivalent with respect to the N-(1-methyl-1H-pyrazol-5-yl)-benzene-1,2-diamine (and hence the reaction product). In some embodiments, the phosphoric acid is provided in a molar excess relative to the N-(1-methyl-1H-pyrazol-5-yl)-benzene-1,2-diamine, such as at least about 1, 1.05, 1.1, 1.5, 2, 2.5, 3, 3.5, 4, 4.5, 5 equivalents or more. The molar equivalents of the phosphoric acid may be from any of these values to any other of these values, for example, from about 1 to about 5 equivalents phosphoric acid relative to N-(1-methyl-1H-pyrazol-5-yl)-benzene-1,2-diamine.

The phosphate salt of the invention produced in these methods may be crystalline, eg phosphate form 1. However, in some embodiments, the process may further comprise a a step of forming a crystallising solution comprising the phosphoric acid addition salt, which may be carried out according to any such step described herein.

Following the reacting step, the processes typically comprise separating excess solvent and phosphoric acid to provide the phosphate salt. In some embodiments, the separation may be achieved by filtration.

In some embodiments, the process for preparing the phosphate salt of the invention may comprise:
reacting 1-methyl-1H-pyrazol-5-amine and 1-fluoro-2-nitrobenzene in an organic solvent (typically tetrahydrofuran) in the presense of base (typically an alkoxide, such as potassium tert-butoxide) to provide 2-(1-methyl-1H-pyrazol-5-amino)-1-nitrobenzene;
reducing 2-(1-methyl-1H-pyrazol-5-amino)-1-nitrobenzene, typically in the presence of a palladium catalyst (such as palladium on carbon) in a polar solvent (eg a polar aprotic solvent such as ethyl acetate, acetonitrile and the like, or a protic solvent such as methanol and the like), to provide N-(1-methyl-1H-pyrazol-5-yl)-benzene-1,2-diamine; and
reacting N-(1-methyl-1H-pyrazol-5-yl)-benzene-1,2-diamine with formaldehyde in a solvent in the presence of phosphoric acid to provide the phosphate salt of the invention.

### L-Tartaric Acid Addition Salt of 1-methyl-1,4,5,10-tetrahydropyrazolo[3,4-b][1,5]benzodiazepine

In a second aspect, the invention provides an L-tartaric acid addition salt of 1-methyl-1,4,5,10-tetrahydropyrazolo[3,4-b][1,5]benzodiazepine. This salt form may be referred to herein as the L-tartrate salt of the invention.

Typically, the L-tartrate salt of the invention is provided in a crystalline form. The crystalline form may be characterised by XRD. Accordingly, in some embodiments the L-tartrate salt is characterised by the XRD shown in FIG. 10.

The L-tartrate salt of the invention may additionally or alternatively be characterised by its melting point. In some embodiments, the melting point of the L-tartrate salt of the invention is about 181° C.

The L-tartrate salt of the invention may be an anhydrous crystal. The anhydrous nature of this crystal form may be determined by TGA.

The L-tartrate salt of the compound of the invention may remain substantially anhydrous and at purity levels that are substantially unchanged when stored under ambient conditions or under accelerated storage conditions. The accelerated storage conditions may comprise elevated temperature (eg 40° C. or 80° C.) and/or increased relative humidity. In some embodiments, the L-tartrate salt may remain substantially anhydrous upon storage for example for at least 1 week, at elevated temperature (eg 40° C.) and at up to about 60% RH, 70% RH or 75% RH. Typically, the L-tartrate salt will also remain stable under these storage conditions, remaining substantially pure throughout, for example resulting in up to about 2%, 1.5% or 1% degradation products detectable by HPLC. The HPLC may be carried out by any of the techniques described herein.

The L-tartrate salt of the invention may be prepared by any suitable means. Typically, the preparation of the L-tartrate salt of the invention comprises exposing the compound of the invention to L-tartaric acid.

Accordingly, also provided is a process for preparing the L-tartrate salt of the invention, comprising:
preparing a solution comprising 1-methyl-1,4,5,10-tetrahydropyrazolo[3,4-b][1,5]benzodiazepine freebase, a solvent, and L-tartaric acid; and
separating excess solvent and L-tartaric acid from the L-tartrate salt.

In some embodiments, the process further comprises preparing a crystallisation solution of the L-tartrate salt and a minimum volume of a crystallisation solvent to form the crystallisation solution. The crystallisation solution may be allowed to stand under ambient conditions and/or cooled and/or concentrated to allow crystal formation.

Also provided is a process for preparing the L-tartrate salt of the invention comprising reacting N-(1-methyl-1H-pyrazol-5-yl)-benzene-1,2-diamine with formaldehyde in the presence of L-tartaric acid to provide the L-tartaric acid addition salt of the compound of the invention.

The reaction of N-(1-methyl-1H-pyrazol-5-yl)-benzene-1,2-diamine with formaldehyde may occur in any suitable solvent, such as any of the crystallisation solvents described herein. The reaction of N-(1-methyl-1H-pyrazol-5-yl)-benzene-1,2-diamine with formaldehyde may further comprise forming a solution of N-(1-methyl-1H-pyrazol-5-yl)-benzene-1,2-diamine and adding formaldehyde to the solution. The solution may comprise any suitable solvent, such as any of the crystallisation solvents for forming tartrate salts described herein.

The phosphate salt of the invention produced in these methods may be crystalline. However, in some embodiments, the process may further comprise a step of forming a crystallising solution comprising the L-tartaric acid addition salt, which may be carried out according to any such step described herein.

In some embodiments, the process for preparing the L-tartaric salt of the invention may comprise:
reacting 1-methyl-1H-pyrazol-5-amine and 1-fluoro-2-nitrobenzene in an organic solvent (typically tetrahydrofuran) in the presense of base (typically an alkoxide, such as potassium tert-butoxide) to provide 2-(1-methyl-1H-pyrazol-5-amino)-1-nitrobenzene;
reducing 2-(1-methyl-1H-pyrazol-5-amino)-1-nitrobenzene, typically in the presence of a palladium catalyst (such as palladium on carbon) in a polar solvent (eg a polar aprotic solvent such as ethyl acetate, acetonitrile and the like, or a protic solvent such as methanol and the like), to provide N-(1-methyl-1H-pyrazol-5-yl)-benzene-1,2-diamine; and
reacting N-(1-methyl-1H-pyrazol-5-yl)-benzene-1,2-diamine with formaldehyde in a solvent in the presence of L-tartaric acid to provide the L-tartrate salt of the invention.

### Crystalline 1-Methyl-1,4,5,10-tetrahydropyrazolo[3,4-b][1,5]benzodiazepine Freebase

In a third aspect, the invention provides a crystal of the compound of the invention. This crystal may be referred to herein as the freebase crystal of the invention.

Typically, the freebase crystal of the invention is an anhydrous crystal.

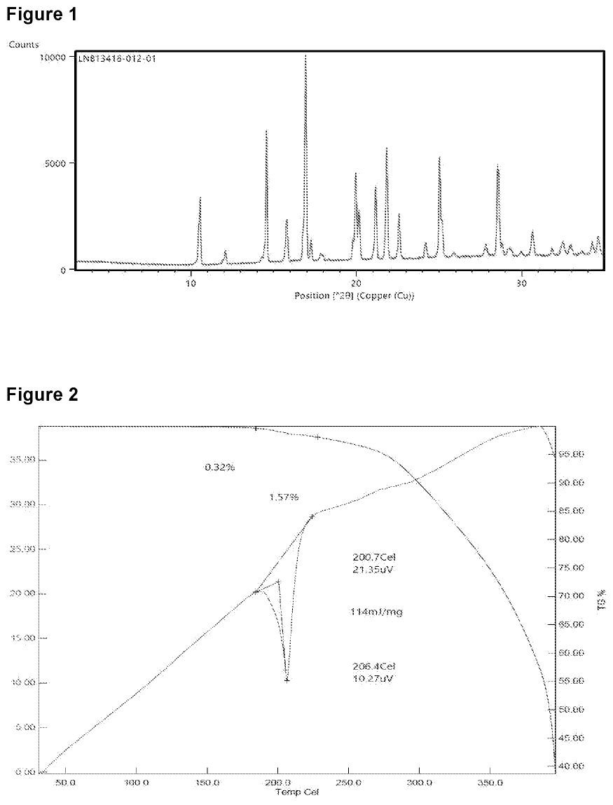

The freebase crystal of the invention may be characterised by its XRD pattern, which is shown in FIG. 1. Additionally or alternatively, the freebase crystal of the invention may be characterised by its melting point, which was determined by DTA and DSC analysis to be about 200° C.

The freebase crystal of the invention may be prepared by any suitable means. Typically, the preparation of the freebase crystal of the invention comprises exposing a salt of the compound of the invention (such as a hydrochloride salt of the compound of the invention) to an aqueous base (such as sodium bicarbonate) to neutralise the acid addition counterion, followed by liquid-liquid extraction with an organic solvent to extract the freebase compound in an organic phase.

Accordingly, also provided is a process for preparing a crystalline form of 1-methyl-1,4,5,10-tetrahydropyrazolo[3,4-b][1,5]benzodiazepine freebase, comprising:
preparing a solution of a hydrochloride salt of 1-methyl-1,4,5,10-tetrahydropyrazolo[3,4-b][1,5]benzodiazepine (for example the dihydrochloride salt) and an aqueous saturated bicarbonate solution; and
contacting the solution with an organic solvent to extract 1-methyl-1,4,5,10-tetrahydropyrazolo[3,4-b][1,5]benzodiazepine freebase from the solution.

Also provided is a process for preparing a crystalline form of 1-methyl-1,4,5,10-tetrahydropyrazolo[3,4-b][1,5]benzodiazepine freebase, comprising providing a solution of 1-methyl-1,4,5,10-tetrahydropyrazolo[3,4-b][1,5]benzodiazepine and allowing the 1-methyl-1,4,5,10-tetrahydropyrazolo[3,4-b][1,5]benzodiazepine to crystalise, wherein the solution is substantially free of acid.

In these methods, the 1-methyl-1,4,5,10-tetrahydropyrazolo[3,4-b][1,5]benzodiazepine may be provided by any suitable means, including by synthesis, including the synthesis described herein.

Also provided is a process for preparing a crystalline form of 1-methyl-1,4,5,10-tetrahydropyrazolo[3,4-b][1,5]benzodiazepine freebase, comprising reacting N-(1-methyl-1H-pyrazol-5-yl)-benzene-1,2-diamine with formaldehyde in a solvent in the presence of an acid, followed by exposing the reaction products to a base, and optional crystalising to provide the crystaline form of 1-methyl-1,4,5,10-tetrahydropyrazolo[3,4-b][1,5]benzodiazepine freebase.

In some embodiments, the acid is acetic acid.

In some embodiments, the base is an aqueous base, such as an aqueous solution of sodium bicarbonate, sodium carbonate, sodium hydroxide, potassium carbonate and the like. The exposing step may comprising multiple washes of the reaction products with the base.

The product following exposure to the base may be in crystaline form, or the process may require a subsequent crystalising step. The crystalising step may comprise providing a solution of 1-methyl-1,4,5,10-tetrahydropyrazolo[3,4-b][1,5]benzodiazepine and allowing the 1-methyl-1,4,5,10-tetrahydropyrazolo[3,4-b][1, 5]benzodiazepine to crystalise. Any suitable crystalising step described herein may be used in these processes.

### Pharmaceutical Compositions

Also provided are medicaments comprising any one or more of the salts and/or crystals of the invention.

Also provided are pharmaceutical compositions comprising any one or more of the salts and/or crystals of the invention. The pharmaceutical compositions typically further comprise a pharmaceutically acceptable carrier, diluent and/or excipient.

The medicaments and pharmaceutical compositions include those for oral, rectal, nasal, topical (including buccal and sub-lingual), parenteral administration (including intramuscular, intraperitoneal, sub-cutaneous and intravenous), or in a form suitable for administration by inhalation or insufflation. The salts and/or crystals of the invention optionally together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for oral use, or in the form of sterile injectable solutions for parenteral (including subcutaneous) use. Typically, the salts and/or crystals of the invention will be employed as solids due to their favourable properties in the solid state.

The pharmaceutical compositions of the salts and/or crystals of the invention may conveniently be presented in dosage unit form and may be prepared by any of the methods well known in the art of pharmacy and may include any conventional carrier, diluent and/or excipient as known in the art of pharmacy (See, for example, Remington: The Science and Practice of Pharmacy, 21st Ed., 2005, Lippincott Williams & Wilkins). Typically, preparation of the pharmaceutical compositions described herein include the step of bringing the active ingredient, for example any one of the salts and/or crystals of the invention, into association with the carrier which constitutes one or more accessory ingredients. In general, the pharmaceutical compositions are prepared by uniformly and intimately bringing the active ingredient, for example salts and/or crystals of the invention, into association with a liquid carrier or a finely divided solid carrier or both, and then, if necessary, shaping the product into the desired formulation. In the pharmaceutical composition the salts and/or crystals of the invention are included in an amount sufficient to produce the desired effect.

The pharmaceutical compositions described herein may be used in any of the methods described herein.

### Methods of Treatment

Methods of treatment involving the compound of the invention are described in WO2017/004674, WO2020/102857 and WO2021/042178. As the salts and/or crystals of the invention are favourable solid forms, possessing low hygroscopicity and improved stability, it is envisaged that they may be used in any of these methods of treatment.

Accordingly, in another aspect there is provided a method of:
treating or preventing antisocial behaviour in a subject, and/or
providing acute and long-term regulation of social behavior in a subject, and/or
treating or preventing a substance abuse disorder in a subject, and/or
treating or preventing a social dysfunction in a subject, and/or
treating or preventing a psychiatric disorder in a subject as part of a therapy for a psychiatric disorder that features social dysfunction as a primary or secondary feature; and/or
causing weight loss in a subject; and/or
managing weight in a subject; and/or
suppressing an appetite for food in a subject; and/or
reducing the consumption of food by a subject; and/or
treating or preventing opioid withdrawal and/or a symptom associated with opioid withdrawal in a subject;
   the method comprising administering to the subject an effective amount of any one or more of the salts and/or crystals of the invention.

In another aspect, also provided is a method of treating a subject suffering from or at risk of developing a substance abuse disorder, or a subject recovering from a substance abuse disorder and seeking to maintain ongoing abstinence of the substance, comprising administering to the subject an effective amount of any one or more of the salts and/or crystals of the invention, to thereby treat or prevent the substance abuse disorder.

In some embodiments, the method of the invention comprises administering an effective amount of a pharmaceutical composition comprising salts and/or crystals of the invention and a pharmaceutically acceptable carrier, diluent and/or excipient.

In some embodiments, the method of treating or preventing antisocial behaviour in a subject comprises stimulating pro-social behaviour in the subject.

In some embodiments, the psychiatric disorder is selected from autism spectrum disorder, substance abuse disorder, schizophrenia, or a combination thereof.

In some embodiments, the substance abuse disorder is selected from addiction and/or dependence on any one of an opioid, an opiate, alcohol, cocaine or a combination thereof.

In some embodiments, the methods of the invention treat a symptom of opioid withdrawal. The symptoms of opioid withdrawal include psychological, physical and/or somatic symptoms.

Physical and somatic symptoms of opioid withdrawal include tremors, shaking, hot or cold flashes, goosebumps, sweating, rapid breathing, elevated heart rate, elevated blood pressure, body aches, vomiting, diarrhea and fever. In some embodiments, methods treat a physical and/or somatic symptom of opioid withdrawal. In some embodiments, the physical and/or somatic symptoms are selected from tremors and shaking.

Psychological symptoms of opioid withdrawal include dysphoria, anxiety, restlessness, irritability, insomnia, yawning, hallucinations, hyperalgesia, hyperkatifiteia, and anorexia. It is believed that although these symptoms are not physical/somatic, they are symptoms of opioid withdrawal and stem from the physiological changes resulting from cessation or reduction of opioid dosing and/or induced by opioid antagonist administration. In some embodiments, the methods treat dysphoria.

Symptoms of opioid withdrawal include dysphoria, anxiety, restlessness, irritability, insomnia, yawning, hallucinations, tremors, shaking, hot or cold flashes, goosebumps, sneezing, sweating, rapid breathing, elevated heart rate, elevated blood pressure, pupillary dilation, piloerection, head aches, body aches, muscle cramps, muscle aches, bone aches, joint aches, hyperalgesia, hyperkatifiteia, watery discharge from eyes and nose (lacrimation and rhinorrhea), nausea, vomiting, diarrhea, abdominal pain, anorexia and fever. As noted above, one of the diagnostic tools developed regarding opioid withdrawal is the DSM-5. The DSM-5 specifies that for a subject to be diagnosed with opioid withdrawal, 3 of the following 9 symptoms must develop within minutes to several days of either cessation (or reduction) of opioid exposure, or the administration of an opioid antagonist or partial agonist. The DSM-5 symptoms are (1) dysphoric mood, (2) nausea, (3) muscle aches, (4) lacrimation or rhinorrhea, (5) pupillary dilation, piloerection or sweating, (6) diarrhea, (7) yawning, (8) fever and (9) insomnia. Accordingly, in some embodiments, the subject experiences at least 1, 2, 3, 4, 5, 6, 7, 8 or 9 of these DSM-5 symptoms and preferably administration of the compound of Formula (I) treats at least one of the symptoms experienced by the subject.

The severity of withdrawal symptoms will depend on the opioid causing the dependence, the dose and length of treatment or abuse, how rapidly opioid use is discontinued and the characteristics of the subject including age, sex, weight etc.

Accordingly, in some embodiments, the methods treat an opioid withdrawal symptom selected from the group consisting of tremors, shaking, hot or cold flashes, goosebumps, sweating, rapid breathing, elevated heart rate, elevated blood pressure, body aches, vomiting, diarrhea, fever, dysphoria, anxiety, restlessness, irritability, insomnia, yawning, hallucinations, hyperalgesia, hyperkatifiteia, and anorexia, or a combination thereof.

### Administration

The salts and/or crystals of the invention may be administered by any suitable means, for example, orally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The salts and/or crystals of the invention may be provided as any suitable dosage form, including any of the medicaments and/or pharmaceutical compositions described herein.

Salts and/or crystals of the invention, may be administered in a dose of about 0.001, 0.005, 0.01, 0.05, 0.1, 0.15, 0.2, 0.5, 1, 2, 3, 5, 10, 15, 20, 25 or 30 mg/kg of the body weight of the subject. In some embodiments, the dose may be from any of these amounts to any other amount, such as from about 0.001 mg/kg to about 30 mg/kg, about 0.2 mg/kg to about 30 mg/kg or about 0.2 mg/kg to about 10 mg/kg. It will be understood, however, that the specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Salts and/or crystals of the invention may be administered in an “effective amount”, for example when an appropriate amount is included in a pharmaceutical composition. “Effective amount” is taken to mean an amount of a compound that will elicit a desired biological or medical response of a tissue, system, animal or human that is being sought by the researcher, veterinarian, medical doctor or other clinician administering the salts and/or crystals of the invention or a composition including the salts and/or crystals of the invention. In some embodiments, the effective amount may be a “therapeutically effective amount” wherein the amount of the salts and/or crystals of the invention is effective to treat the condition and/or symptom thereof that has manifested in the subject. In other embodiments, the effective amount may be a “prophylactically effective amount” wherein the amount of the salts and/or crystals of the invention is sufficient to prophylactically treat and/or prevent the onset of the condition and/or a symptom thereof or, if a symptom emerges, cause the severity of the condition and/or symptom thereof to be at a reduced level compared to the average severity of the condition and/or symptom thereof in a population of subjects not having received treatment with the compound of Formula (I) and/or a pharmaceutically acceptable salt and/or prodrug thereof.

The “effective amount” will be dependent on a number of factors, including the physical condition of the subject to be treated, the severity of symptoms, the formulation of the compound, and/or a professional assessment of the medical situation. The subject's weight and age may also be a factor for the person skilled in the art when determining the amount of salts and/or crystals of the invention that the subject should receive.

The phrases “administration of” and or “administering a” salt and/or crystal of the invention should be understood to mean providing the object active compound to a subject in need thereof.

As provided herein, beneficial or desired clinical results from the disclosed salt and/or crystal of the invention, include, without limitation, cessation of a symptom of the object disease, disorder or condition; alleviation of severity of a symptom of the object disease, disorder or condition; prevention of onset of a symptom of the object disease, disorder or condition; and/or managing a symptom of the object disease, disorder or condition for example preventing worsening of severity of a symptom or causing the symptom to reduce in severity or cease within a shorter than expected time. Either therapeutic or preventative measures may be achieved. Those in need of treatment include those already experiencing the object disease, disorder or condition as well as those in which the object disease, disorder or condition is to be prevented. By treatment is meant inhibiting or reducing an increase in symptoms of the object disease, disorder or condition when compared to the absence of treatment, and is not necessarily meant to imply complete cessation of the relevant condition.

Thus, generally, the term “treatment” (and variations thereof including “treating”) means affecting a subject, tissue or cell to obtain a desired pharmacological and/or physiological effect, including the beneficial or desired clinical results discussed above.

### Kits

Also provided is a kit of parts, comprising in separate parts:
one or more of the salts and/or crystals of the invention; and
instructions for its use in any of the methods of the invention.

In any of the kits disclosed herein, the salts and/or crystals of the invention may be formulated as a pharmaceutical composition optionally together with a pharmaceutically acceptable carrier, diluent and/or excipient. The pharmaceutical compositions may be formulated for administration by any route disclosed herein including for oral, rectal, nasal, topical (including buccal and sub-lingual), parenteral administration (including intramuscular, intraperitoneal, sub-cutaneous and intravenous), or in a form suitable for administration by inhalation or insufflation.

### EXAMPLES

### General Methods

### X-Ray Powder Diffraction (XRPD)

XRPD analysis was carried out on a PANalytical X'pert pro with PIXcel detector (128 channels), scanning the samples between 3 and 35° 2θ. The material was gently ground to release any agglomerates and loaded onto a multi-well plate with Kapton or Mylar polymer film to support the sample. The multi-well plate was then placed into the diffractometer and analysed using Cu K radiation (α1 λ=1.54060 Å; α2=1.54443 Å; β=1.39225 Å; α1: α2 ratio=0.5) running in transmission mode (step size 0.0130° 20, step time 18.87 s) using 40 kV/40 mA generator settings. Data were visualized and images generated using the HighScore Plus 4.7 desktop application (PANalytical, 2017).

### Polarised Light Microscopy (PLM)

The presence of crystallinity (birefringence) was determined using an Olympus BX50 microscope, equipped with cross-polarising lenses and a Motic camera. Images were captured using Motic Images Plus 2.0. All images were recorded using the 20× objective, unless otherwise stated.

### Thermogravimetric/Differential Thermal Analysis (TGA/DTA)

Approximately, 5 mg of material was weighed into an open aluminium pan and loaded into a simultaneous thermogravimetric/differential thermal analyser (TG/DTA) and held at room temperature. The sample was then heated at a rate of 10° C./min from 20° C. to 300° C. during which time the change in sample weight was recorded along with any differential thermal events (DTA). Nitrogen was used as the purge gas, at a flow rate of 300 cm³/min.

### Differential Scanning Calorimetry (DSC)

Approximately, 5 mg of material was weighed into an aluminium DSC pan and sealed nonhermetically with a pierced aluminium lid. The sample pan was then loaded into a Seiko DSC6200 (equipped with a cooler) cooled and held at 20° C. Once a stable heat-flow response was obtained, the sample and reference were heated to 250° C. at scan rate of 10° C./min and the resulting heat flow response monitored. Nitrogen was used as the purge gas, at a flow rate of 50 cm³/min.

### Nuclear Magnetic Resonance (NMR)

NMR experiments were performed on a Bruker AVIIIHD spectrometer equipped with a DCH cryoprobe operating at 500.12 MHz for protons. Experiments were performed in deuterated DMSO and each sample was prepared to about 10 mM concentration.

### Dynamic Vapour Sorption (DVS)

Approximately, 10-20 mg of sample was placed into a mesh vapour sorption balance pan and loaded into a DVS Intrinsic dynamic vapour sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH. Two cycles were performed. The weight change during the sorption/desorption cycles were plotted, allowing for the hygroscopic nature of the sample to be determined. XRPD analysis was then carried out on any solid retained.

### Gravimetric Vapour Sorption (GVS)

Approximately 10-20 mg of sample was placed into a mesh vapour sorption balance pan and loaded into an IGASorp Moisture Sorption Analyser balance by Hiden Analytical. The sample was subjected to a ramping profile from 40-90% relative humidity (RH) at 10% increments, maintaining the sample at each step until a stable weight had been achieved (98% step completion, minimum step length 30 minutes, maximum step length 60 minutes) at 25° C. After completion of the sorption cycle, the sample was dried using the same procedure to 0% RH, and finally taken back to the starting point of 40% RH. Two cycles were performed. The weight change during the sorption/desorption cycles were plotted, allowing for the hygroscopic nature of the sample to be determined.

### High Performance Liquid Chromatography-Ultraviolet Detection (HPLC-UV)

Instrument: Dionex Ultimate 3000
Column: Agilent SB-Phenyl 150 mm×4.6 mm, 3.5 μm
Column Temperature: 30° C.
Autosampler Temperature: 5° C.
UV wavelength: 275 nm
Injection Volume: 3 μl
Flow Rate: 1 ml·min-1
Mobile Phase A: 0.1% TFA in 95-5 water: MeCN
Mobile Phase B: 0.1% TFA in MeCN
Gradient program:

| Time (minutes) | Solvent B [%] |
|---|---|
| 0 | 0 |
| 13.0 | 55 |
| 14.0 | 55 |
| 14.1 | 0 |
| 20 | 0 |

Liquid Chromatography-Mass Spectrometry
Instrument: Dionex Ultimate 3000
Thermo Finnigan LCQ Advantage MS
Column: Ace Excel 2 C18-PFP, 75 mm×4.6 mm, 2 μm
Column Temperature: 30° C.
Autosampler Temperature: 5° C.
UV wavelength: 275 nm
Injection Volume: 10 μl
Flow Rate: 1 ml·min-1
Mobile Phase A: 0.1% formic acid in water
Mobile Phase B: 0.1% formic acid in MeCN
Gradient program:

| Time (minutes) | Solvent B [%] |
|---|---|
| 0 | 5 |
| 12.0 | 95 |
| 15.0 | 95 |
| 15.1 | 5 |
| 20 | 5 |

### Example 1—Preparation of Freebase of 1-methyl-1,4,5,10-tetrahydropyrazolo[3,4-b][1,5]benzodiazepine from its Di-Hydrochloride Salt

1-Methyl-1,4,5,10-tetrahydropyrazolo[3,4-b][1, 5]benzodiazepine freebase was prepared for use in a primary salt screen (Example 2) using the following procedure:
About 5.5 g of di-hydrochloride salt of 1-methyl-1,4,5,10-tetrahydropyrazolo[3,4-b][1,5]benzodiazepine was added to a 1 L round bottom flask and mixed with 200 mL of saturated NaHCO₃ solution prepared in deionised water.
500 mL of dichloromethane (DCM) and 200 mL of ethyl acetate (EtOAc) were added to the flask.
The mixture was mixed vigorously, and the organic phase separated.
The aqueous phase was extracted with an additional 200 mL of DCM.
The separated organic phases were combined and dried over Na₂SO₄ and filtered.
The filtered solution was concentrated in vacuo to give a beige solid.
The solid was triturated with tert-butyl methyl ether (TBME) before filtration and drying on the filter bed (eg for about 1 hour). The material may be optionally be further dried under reduced pressure at about 40° C.

The isolated 1-methyl-1,4,5,10-tetrahydropyrazolo[3,4-b][1,5]benzodiazepine freebase was characterized by TG/DTA, DSC, DVS with post-DVS, XRPD, HPLC, ¹H NMR and LC-MS, as per the methods detailed above. The 1-methyl-1,4,5,10-tetrahydropyrazolo[3,4-b][1,5]benzodiazepine freebase was stored at 40° C./75% relative humidity (RH) for 1 week to determine the stability of the material at increased RH and to identify potential hydrate formation.

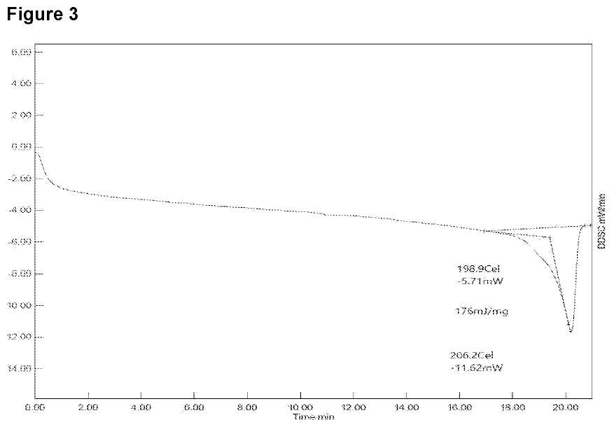

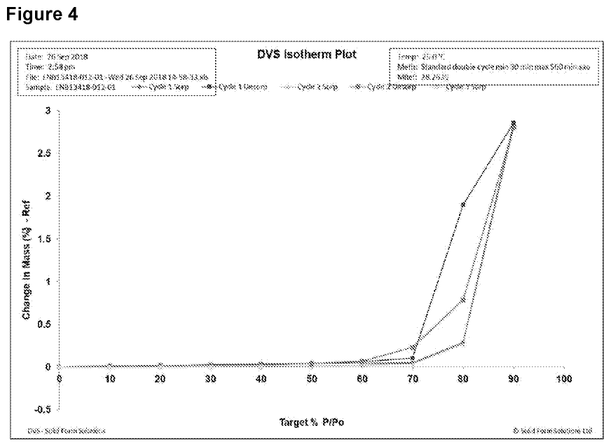

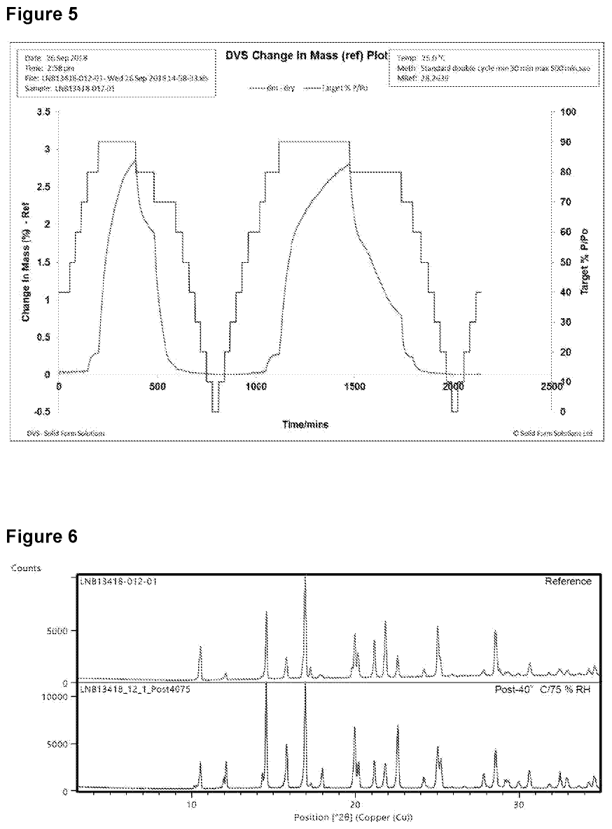

The following results were obtained during the preparation of the freebase:
3.627 g of freebase was isolated.
The isolated material appeared highly crystalline by XRPD (FIG. 1).
The material appeared anhydrous by TG analysis with no significant mass loss observed. A mass loss of about 1.6% was observed during the observed melting transition, likely due to solvent trapped in the crystalline material (about 0.12 equivalents of EtOAc, 0.12 equivalents of DCM, or 0.6 equivalents of water) (FIG. 2).
DT analysis showed a large, sharp melting point from an onset of about 201° C., with a peak at 206° C. No other thermal events were observed (FIG. 2).
DSC analysis showed a large melting endotherm from an onset of about 199° C., with a peak at about 206° C., corresponding to the melting transition observed in the DT analysis (FIG. 3). No thermal events were observed during the cool and second heat of the DSC analysis.
The material appeared hygroscopic by DVS above 70% RH with a mass increase of about 2.74% between 70 and 90% RH (about 0.31 equivalents of water). Between 0 and 70% RH the material was non-hygroscopic with a mass increase of about 0.11% (FIG. 4 and FIG. 5). No change in form was observed by XRPD post-DVS analysis.
The ¹H NMR spectrum showed the expected connectivity of the structure. Traces of EtOAc and DCM were observed.
HPLC analysis confirmed the material exhibited a high purity (by area %) of 98.4%.
LC-MS showed a mass of 201.03 m/z positive ionization[M+H], corresponding to the expected mass of 200.24 g/mol.
No significant changes in pattern were observed post-storage at 40° C./75% RH for 1 week. The material appeared more crystalline by XRPD compared to XRPD of a sample prior to 1-week stability testing. New small peaks were observed at about 10.2, 11.9 and 14.3° 2θ, related to the improvement in crystallinity or a potential hydrate beginning to form (FIG. 6).

The freebase of the compound of the invention was successfully produced and fully characterized. The material was found to be highly crystalline by XRPD. The material appeared anhydrous by TG analysis. DTA and DSC analysis confirmed a melting point of about 200° C. the freebase appeared hygroscopic by DVS with a mass increase of about 2.74% between 70 and 90% RH. Between 0 and 70% RH the material appears non-hygroscopic with a mass increase of about 0.11%. No change in form was observed by XRPD post-DVS analysis. The collected ¹H NMR spectrum showed the expected connectivity of the structure provided. A high purity of 98.4% was confirmed b HPLC analysis. LC-MS showed a m/z of 201.3, corresponding to the expected mass of 200.24 g/mol. The freebase material stored at 40° C./75% RH for 1-week showed no changes in form by XRPD.

### Example 2—Primary Salt Screen

A salt screen was conducted on 1-methyl-1,4,5,10-tetrahydropyrazolo[3,4-b][1,5]benzodiazepine freebase using 5 solvent systems and 18 acid counterions (see Table 1).

The solvent systems used in this salt screen were (1) ethanol (EtOH); (2) tetrahydrofuran (THF); (3) isopropyl acetate; (4) acetone; and (5) 95% 2-propanol, 5% water (% v/v).

The following procedure was used:
Approximately 30 mg of 1-methyl-1,4,5,10-tetrahydropyrazolo[3,4-b][1,5]benzodiazepine freebase was weighed into 5×1.5 mL glass vials.
300 μL of selected solvent system was added to each vial to form a mobile slurry, and a beige slurry was observed.
1.05 (or 0.525 for the hemi experiments) equivalents of acid counterion (see Table 1) was added to each sample and initial observations were recorded.
The samples were temperature cycled between ambient and 40° C. in 4-hour cycles for about 72 hours.
The samples were collected, and observations made.
Samples in which solids were observed were filtered via centrifugation and the solids were loaded onto a multi-well XRPD plate and analyzed by XRPD.
Samples in which no solids were observed were uncapped and left undisturbed to allow evaporation at ambient temperature. Brown gums were observed in all samples post-evaporation.
The XRPD plate was placed in an oven at 40° C. for about 24 hours. The dried samples analyzed by XRPD to identify any changes in pattern/potential anhydrous salts.
The XRPD plate was then placed in a stability chamber at 40° C./75% RH for about 24 hours.
The post-stability samples were analyzed by XRPD to identify potential hydrate formation and disproportionation of salts.
Observed patterns which were found to be stable upon drying and storage on 40° C./75% RH were analyzed by TG/DTA to identify salt forms suitable for scale up.

During the primary salt screen of the 18 acid counterions tested, 16 produced potential solid salt forms of the compound. However, only the phosphate and L-tartrate salts possessed suitable properties based on thermal analysis and stability of the observed patterns at 40° C./75% RH.

TABLE 1
Acid counterions, preparation of stock solutions and amounts added to salt
screen samples

| | | Acid Stock | | Known | Volume | Neat Addition | | |
|---|---|---|---|---|---|---|---|---|
| | | Solution Maker | | Suitable | of stock | Amounts | | |
| | No. of | Amount of | ml of | Stock | Solution | Mass of | Volume of | Other |
| Acid | Equivalents | acid | solvent | Solvent | (μL) | Acid (mg) | Acid (μL) | Comments |
| Hydrobromic acid | 1.05 | 565.6 μl | 4434.4 | THF | 157.3 | 26.52 | 17.8 | — |
| 1-5-Naphthalene | 0.525 | 1843.5 mg | 5000 | Neat | 78.7 | 29.00 | — | add 1 eq HCl |
| disulfonic acid | | | | | | | | |
| sodium salt | | | | | | | | |
| Sulfuric acid | 0.525 | 272.0 μl | 4728.0 | THF | 78.7 | 7.87 | 4.3 | — |
| 1-2-Ethane | 0.525 | 1194.6 mg | 5000 | Neat | 78.7 | 18.79 | — | add 1 eq HCl |
| disulfonic acid | | | | | | | | |
| sodium salt | | | | | | | | |
| 1-5-Naphthalene | 1.05 | 1843.5 mg | 5000 | Neat | 157.3 | 116.00 | — | add 2 eq HCl |
| disulfonic acid | | | | | | | | |
| sodium salt | | | | | | | | |
| Sulfuric acid | 1.05 | 271.96 μl | 4728.0 | THF | 157.3 | 31.49 | 17.1 | — |
| 1-2-Ethane | 1.05 | 1194.6 mg | 5000 | Neat | 157.3 | 75.17 | — | add 2 eq HCl |
| disulfonic acid | | | | | | | | |
| sodium salt | | | | | | | | |
| Ethane sulfonic acid | 1.05 | 429.4 μl | 4570.6 | THF | 157.3 | 18.24 | 13.5 | — |
| p-Toluene sulfonic | 1.05 | 980.5 mg | 5000 | EtOH | 157.3 | 30.85 | — | — |
| acid | | | | | | | | |
| Methane sulfonic | 1.05 | 327.7 μl | 4672.3 | THF | 157.3 | 15.3 | 10.3 | — |
| acid | | | | | | | | |
| Naphthalene-2- | 1.05 | 1644.4 mg | 5000 | Neat | 157.3 | 51.74 | — | add 1 eq HCl |
| sulfonic acid | | | | | | | | |
| sodium salt | | | | | | | | |
| Benzene sulfonic | 1.05 | 807.0 mg | 5000 | THF | 157.3 | 25.39 | — | — |
| acid | | | | | | | | |
| 2-Hydroxy | 1.05 | 755.7 mg | 5000 | Neat | 157.3 | 23.77 | — | add 1 eq HCl |
| ethanesulfonic | | | | | | | | |
| acid sodium salt | | | | | | | | |
| Maleic acid | 1.05 | 586.2 mg | 5000 | THF | 157.3 | 18.44 | — | — |
| Phosphoric acid | 1.05 | 341.1 μl | 4658.9 | THF | 157.3 | 18.14 | 10.7 | — |
| Malonic acid | 1.05 | 525.6 mg | 5000 | THF | 157.3 | 16.54 | — | — |
| 2-5- | 1.05 | 786.3 mg | 5000 | THF | 157.3 | 24.74 | — | — |
| Dihydroxybenzoic | | | | | | | | |
| acid | | | | | | | | |
| L-Tartaric acid | 1.05 | 754.2 mg | 5000 | THF | 157.3 | 23.73 | — | — |
| Hydrochloric acid | 1.05 | 417.5 μl | 4582.5 | THF | 157.3 | 15.50 | 13.1 | — |
| 37 wt % (12M) | | | | | | | | |

Results of this salt screen are summarized in table 2.

TABLE 2
Summary of results of acid addition salt screen

| | | | | | Post | | | |
|---|---|---|---|---|---|---|---|---|
| | | | Salt | Post- | 40° C./ | | Solvated/ | |
| Salt | Pattern | Solvent | Stoichiometry | Drying | 75% RH | TG/DTA | hydrated | Comments |
| Hydrobromide | 1 | Ethanol | Mono | Pattern 1 | Pattern 1 | Solvent | Yes | Good |
| | | | | | | loss about | | stability. No |
| | | | | | | 82° C. | | defined |
| | | | | | | potential | | melting |
| | | | | | | solid-solid | | point |
| | | | | | | transition | | observed, |
| | | | | | | about | | however |
| | | | | | | 186° C. | | the material |
| | | | | | | exothermic | | appears |
| | | | | | | degradation | | thermally |
| | | | | | | about | | stable. |
| | | | | | | 252° C. | | |
| | 2 | THF, | | Pattern 2 | Pattern 2 | Solvent | Yes | |
| | | IPAC, | | | | loss about | | |
| | | acetone, | | | | 54° C. | | |
| | | 95% IPA: | | | | potential | | |
| | | 5% H2O | | | | solid-solid | | |
| | | (% v/v) | | | | transition | | |
| | | | | | | about | | |
| | | | | | | 174° C. | | |
| | | | | | | exothermic | | |
| | | | | | | degradation | | |
| | | | | | | about | | |
| | | | | | | 250° C. | | |
| Sulfate | 1 | EtOH | Mono | Pattern 1 | Pattern 4 | n/a | Yes | Complex |
| | 2 | Acetone, | | Pattern 2 | Pattern 2 | Solvent | Unknown | crystalline |
| | | 95% | | | | loss | | profile |
| | | IPA: | | | | about | | |
| | | 5% | | | | 55° C. | | |
| | | H2O | | | | Melting | | |
| | | (% v/v) | | | | point | | |
| | | | | | | about | | |
| | | | | | | 189° C. | | |
| | 3 | Ethanol + | Hemi | Pattern 3 | Pattern 3 | n/a | Unknown | |
| | | 95% IPA: | | | | | | |
| | | 5% H2O | | | | | | |
| | | (% v/v) (hemi | | | | | | |
| | | experiments) | | | | | | |
| | 4 | n/a | Mono | n/a | n/a | Thermal | Yes | |
| | | | | | | degradation | | |
| | | | | | | about | | |
| | | | | | | 182° C. | | |
| Mesylate | 1 | 95% IPA: 5% | Mono | Pattern 1 | Pattern 1/2 | n/a | Unknown | Complex |
| | | H2O (% v/v) | | | | | | crystalline |
| | 2 | IPAC | | Pattern 2 | Pattern 1 | | | profile |
| | 3 | Acetone | | Pattern 3 | Deliquesced | | | |
| Phosphate | 1 | Ethanol, | Mono | Pattern 1 | Pattern 1 | Melting | No | Good |
| | | THF, IPAC, | | | | point about | | stability |
| | | acetone, | | | | 207° C. | | |
| | | 95% IPA: | | | | | | |
| | | 5% H2O | | | | | | |
| | | (% v/V) | | | | | | |
| Esylate | 1 | THF, IPAC, | Mono | Pattern 1 | Pattern 2 | Solvent loss | No | Hydrated |
| | | acetone | | | | about 75° C. | | |
| | | | | | | Possible solid-solid | | |
| | | | | | | transition | | |
| | | | | | | about | | |
| | | | | | | 131° C. Melt/ | | |
| | | | | | | degradation | | |
| | | | | | | about 216° C. | | |
| | 2 | 95% IPA: | | Pattern 2 | Pattern 2 | Solvent loss | Yes | |
| | | 5% H2O | | | | about | | |
| | | (% v/v) | | | | 100° C. | | |
| | | | | | | Exothermic | | |
| | | | | | | degradation | | |
| | | | | | | about | | |
| | | | | | | 227° C. | | |
| Tosylate | 1 | Ethanol | Mono | Pattern 1 | Pattern 1 | Melting | No | Good |
| | | | | | | point about | | stability. |
| | | | | | | 189° C. | | Forms |
| | | | | | | | | hydrates/ |
| | | | | | | | | solvates. |
| | 2 | THF | | Pattern 2 | Pattern 2 | Solvent loss | Yes | |
| | | | | | | about 61° C. | | |
| | | | | | | Melting | | |
| | | | | | | point about | | |
| | | | | | | 179° C. | | |
| | 3 | IPAC, | | Pattern 3 | Pattern 3 | Solvent loss | Yes | |
| | | acetone, | | | | about 77° C. | | |
| | | 95% IPA: | | | | Melting | | |
| | | 5% H2O | | | | point about | | |
| | | (% v/v) | | | | 203° C. | | |
| Besylate | 1 | THF, IPAC | Mono | Pattern 1 | Pattern 1, 2 | n/a | Pattern 2 and | Complex |
| | | and acetone | | | and 3 | | 3-hydrated | crystalline |
| | | | | | | | | profile |
| Maleate | 1 | THF and | Mono | Pattern 1 | Pattern 1 | Melting | No | Low melting |
| | | IPAC | | | | point about | | point |
| | | | | | | 137° C. | | |
| | | | | | | Second | | |
| | | | | | | melting | | |
| | | | | | | point about | | |
| | | | | | | 146° C. | | |
| Napsylate | | THF | Mono | Pattern 1 | Pattern 2 | Thermal degradation | Pattern | Good |
| | | | | | | about 169° C. | 2-hydrated | stability. |
| | | | | | | | | Hydrate |
| | | | | | | | | formation |
| | | | | | | | | observed. |
| | 2 | IPAC | | Pattern 2 | Pattern 2 | n/a | | |
| Napadisylate | 1 | Ethanol, | Mono | Pattern 1 | Pattern 1 | Thermal degradation | No | Good |
| | | THF, IPAC, | | | | about 171° C. | | stability. No |
| | | acetone, | | | | | | defined |
| | | 95% IPA: | | | | | | melting |
| | | 5% H2O | | | | | | point |
| | | (% v/v) | | | | | | observed, |
| | | | | | | | | however the |
| | | | | | | | | material |
| | | | | | | | | appears |
| | | | | | | | | thermally |
| | | | | | | | | stable. |
| | 2 | Ethanol, | Hemi | Pattern 2 | Pattern 2 | Solvent loss | | Solvent loss |
| | | THF | | | | about | | may indicate |
| | | acetone, | | | | 142° C. | | stable |
| | | 95% IPA: | | | | Thermal | | hydrate |
| | | 5% H2O | | | | degradation | | |
| | | (% v/v) | | | | about 239° C. | | |
| Edisylate | 1 | Ethanol, | Mono | Pattern 1 | Pattern 2 | Solvent loss | | Good |
| | | THF, IPAC, | | | | from onset | | stability. |
| | | acetone, | | | | of heating. | | Stability of |
| | | 95% IPA: | | | | Thermal | | the hydrate |
| | | 5% H2O | | | | | | should be |
| | | (% v/v) | | | | | | investigated |
| | | | | | | degradation | Pattern 2- | |
| | | | | | | about 164° C. | hydrated | |
| | 2 | Ethanol | Hemi | Pattern 2 | Pattern 2 | Solvent loss | | |
| | | | | | | about 48° C. | | |
| | | | | | | Thermal | | |
| | | | | | | degradation | | |
| | | | | | | about | | |
| | | | | | | 175° C. | | |
| Isethionate | 1 | Ethanol | Mono | Pattern 1 | Pattern 3 | Solvent loss | Pattern 3- | All patterns |
| | | | | | | about 60° C. | hydrated | have good |
| | | | | | | melting point | | stability. |
| | | | | | | about 190° C. | | Hydrate |
| | | | | | | | | formation |
| | | | | | | | | observed |
| | 2 | THF, IPAC, | | Pattern 2 | Pattern 3 | Solvent loss | | |
| | | acetone, | | | | about 54° C. | | |
| | | 95% IPA: | | | | melting | | |
| | | 5% H2O | | | | point about | | |
| | | (% v/v) | | | | 190° C. | | |
| | 3 | n/a | | n/a | n/a | Solvent loss | | |
| | | | | | | about 59º C. | | |
| | | | | | | melting | | |
| | | | | | | point about | | |
| | | | | | | 188° C. | | |
| Tartrate | 1 | Ethanol, | Mono | Pattern 1 | Pattern 1 | Melting | No | Good |
| | | THF, IPAC, | | | | point about | | stability |
| | | acetone, | | | | 178° C. | | |
| | | 95% IPA: | | | | | | |
| | | 5% H2O | | | | | | |
| | | (% v/v) | | | | | | |

From the above results, only the phosphate and L-tartrate salts possessed acceptable thermal stability and hygroscopicity.

During the primary polymorph screen, 16 potential salt forms were identified. Potential salt forms which appeared stable at 40° C./75% RH were analyzed by TG/DTA to identify salt forms with desirable thermal properties. TG/DTA analysis identified 4 potential salt forms with desirable properties; phosphate pattern 1 (phosphate form 1), L-tartrate pattern 1 (L-tartrate form 1), tosylate pattern 1 and tosylate pattern 3. Thermal analysis showed the phosphate form 1 and L-tartrate form 1 to be anhydrous.

### Example 3—Preparation of Phosphate Salt of 1-methyl-1,4,5,10-tetrahydropyrazolo[3,4-b][1,5]benzodiazepine form 1

This example describes protocols to prepare phosphoric acid addition salts of 1-methyl-1,4,5,10-tetrahydropyrazolo[3,4-b][1,5]benzodiazepine.

Experiment 3.1—Preparation from Freebase

The 1-methyl-1,4,5,10-tetrahydropyrazolo[3,4-b][1,5]benzodiazepine phosphate form 1 was prepared using the following procedure:
Approximately 500 mg of 1-methyl-1,4,5,10-tetrahydropyrazolo[3,4-b][1,5]benzodiazepine freebase was weighed into a 20 mL scintillation vial.
5 mL of ethanol was added to the material to form a mobile slurry.
2.62 ml (1.05 equivalents) of 1M phosphoric acid stock solution (prepared in THF) was added to the material slurry. A pink slurry was observed upon addition of the acid counterion.
The slurry was then temperature cycled between ambient and 40° C. in 4-hours cycles with stirring for about 24 hours. After about 2 hours stirring at 40° C. a thick pink slurry was observed.
The sample was collected after about 24 hours, and a small amount of material was isolated using a plastic pipette and analyzed by XRPD whilst damp.
The bulk material was filtered using a Buchner funnel and dried on the filter bed for about 1 hour. The material filtered rapidly and a flowable, pink powder was observed.
The bulk material was dried under vacuum at 40° C. for about 16 hours.

The dried material was fully characterised by TG/DTA, DSC, DVS with post-DVS, XRPD, HPLC, ¹H NMR and LC-MS, as per the methods detailed above.

The isolated material appeared highly crystalline by XRPD (FIG. 7).
PLM micrographs show small, birefringent particles with no defined morphology (not shown). The lack of morphology may be due to the use of stirrer bars in the scale up process.
TG analysis showed no mass loss until thermal degradation above about 200° C. (FIG. 8).
DT analysis showed a large endothermic melting transition from an onset of about 209° C. with a peak at 214° C. (FIG. 8).

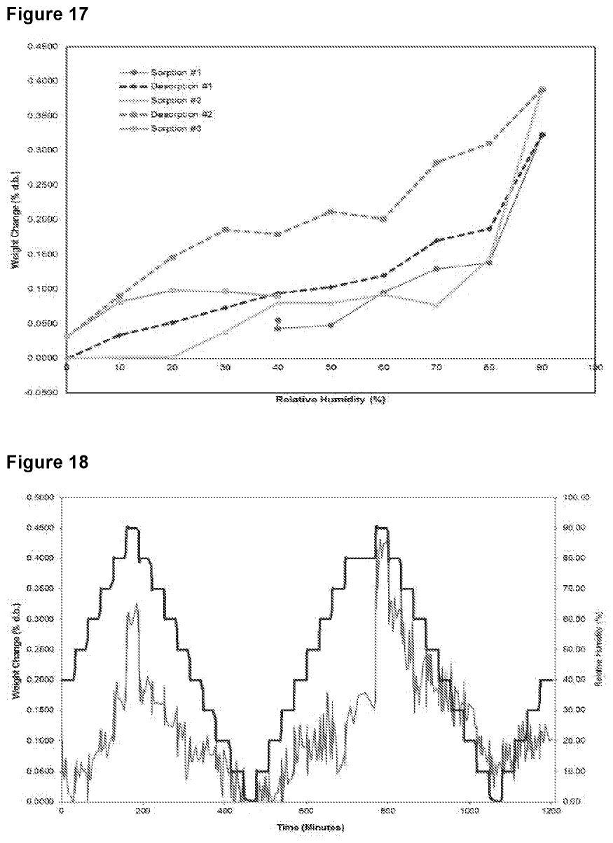

Phosphate form 1 was successfully prepared by the above process. The isolated material appeared highly crystalline by XRPD and PLM. The observed crystals showed no defined morphology by PLM which may be due to the use of a stir-bar during preparation. TG analysis confirmed the material was anhydrous. DT and DSC analysis identified a melting point of about 200° C., consistent with the primary screen data. The material appeared slightly hygroscopic by DVS with a mass increase of about 0.4% at 90% RH (FIGS. 17 and 18). No evidence of form changes or hydrate formation was observed by XRPD post-DVS analysis. The collected ¹H NMR spectrum showed the expected connectivity with the structure provided. The quantitative ³¹P NMR confirmed the presence of 1 equivalent of phosphorus in the material. HPLC analysis confirmed a high purity of 98.7% (by area %), showing a small uplift in purity from the freebase input material.

### Experiment 3.2—Preparation by Synthesis

The 1-methyl-1,4,5,10-tetrahydropyrazolo[3,4-b][1,5]benzodiazepine phosphate form 1 was prepared by reacting N-(1-methyl-1H-pyrazol-5-yl)-benzene-1,2-diamine with formaldehyde in the presence of phosphoric acid. N-(1-methyl-1H-pyrazol-5-yl)-benzene-1,2-diamine may be prepared as described in Katte, TA; Reekie, TA; Jorgensen, WT; and Kassiou, M; J. Org. Chem. 2016, 81(11), 4883-4889, which is entirely incorporated by reference including all supporting information.

N-(1-methyl-1H-pyrazol-5-yl)-benzene-1,2-diamine was recrystallised from ethyl acetate and heptane prior to inclusion in the following reaction. The recrystalised N-(1-methyl-1H-pyrazol-5-yl)-benzene-1,2-diamine had a purity of 99.4% area assessed by HPLC using conventional separation techniques. It will be appreciated that recrystalisation is not essential for successful completion of the following reaction with formaldehyde.

A 1 litre flask was fitted with a condenser, pressure equalising addition funnel, nitrogen inlet and magnetic stirrer bar and purged with nitrogen. Recrystalised N-(1-methyl-1H-pyrazol-5-yl)-benzene-1,2-diamine (37.2 g) was added to the flask, followed by a mixture of acetonitrile (184.6 g) and water (145.0 g) which had been previously degassed. The mixture was heated to 30° C., and shortly after 2.9 g of a mixture of phosphoric acid (23.1 g) and water (26.3 g) added, washing in with water (14.9 mL). After 10 minutes, 37% aqueous formaldehyde (15.9 g) was added over 25 minutes maintaining the temperature between 36° C. and 43° C. The solution was washed in with water (14.9 mL) within 10 minutes. Seventy (70) minutes after formaldehyde addition began, the mixture was filtered into a nitrogen purged receiver, and the reaction flask washed out with acetonitrile (7.9 mL), the wash being passed through the filter. The liquor flask was purged with nitrogen, and reheated to 34° C. The remaining phosphoric acid was diluted further using acetonitrile (49.2 g) and added over 35 minutes at 34° C. to 39° C., washing in with acetonitrile (5.8 g). The mixture was stirred for 5 minutes, then cooled to 19° C. over 2 hours, then cooled in an ice bath for 105 minutes. At 6° C., the mixture was filtered, the cake washed with approximately two thirds of a mix of acetonitrile (26.4 g) and water (22.8 g), then the cake compressed. The filter cake was washed with the remaining third of the acetonitrile/water mixture, dried for 2 minutes on the sinter with a nitrogen blanket. The pale pink solid (66.9 g) was transferred to an oven and dried at 40° C. for 115 hours to provide Phosphate Form 1 (Yield 51.0 g, 86.5%).

### Example 4—Polymorph Screen of Phosphate Salt and Preparation of Phosphate Salt of 1-methyl-1,4,5,10-tetrahydropyrazolo[3,4-b][1,5]benzodiazepine Pattern 2

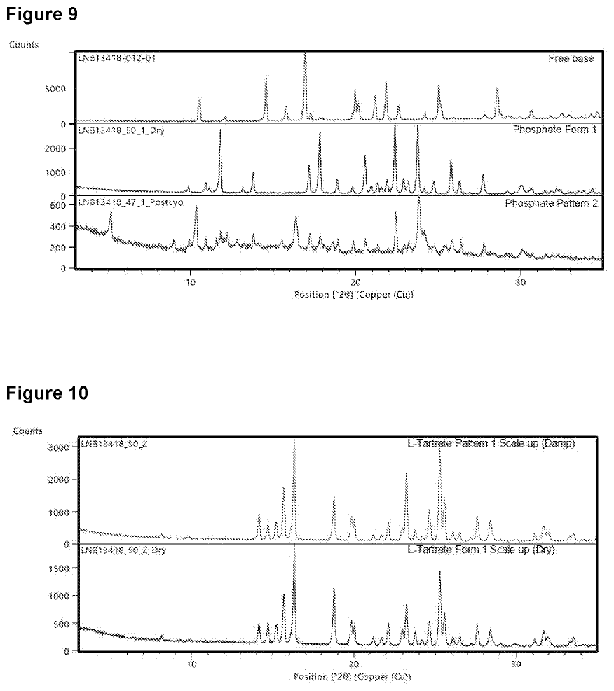

Lyophilized material of the phosphoric acid of the compound of the invention was prepared using the following procedure:
Approximately 1 g of freebase was weighed and added to a 100 mL round bottom flask.
15 mL of 1,4-dioxane was added to form a mobile slurry.
1.05 equivalents (357.7 μL) of neat phosphoric acid was added. Upon agitation a pink slurry was observed.
60 mL of deionized water was added to the flask and the mixture was heated gently until dissolution was observed.
The resultant clear, pink solution was split between 25×20 mL vials and the samples were frozen. Each vial contained about 40 mg of phosphate salt.
The samples were then lyophilized over about 72 hours.
Post-lyophilization a pale pink, fluffy solid was observed.
XRPD analysis of the material showed a poorly crystalline pattern which had not been previously observed. The pattern was denoted phosphate pattern 2 (differences in XRPD patterns for freebase, form 1 and pattern 2 are shown in FIG. 9).

### Solvent Solubility

Selected solvent was added in 50 μL aliquots to approximately 10 mg of poorly crystalline phosphate pattern (obtained from above lyophilisation). Between each addition, the mixture was checked for dissolution and where no dissolution was apparent, the mixture was heated to about 40° C. and checked again. This procedure was continued until dissolution was observed or until 2 mL of solvent had been added. Samples in which dissolution was observed were stored uncapped at ambient temperature to allow evaporation. Samples in which dissolution was not observed were filtered via centrifugation. All observed solids were analyzed by XRPD. The solvent systems used during the solvent solubility screen are detailed in Table 3.

During the solvent solubility screen the following results were obtained:
Low solubility (<5 mg/mL) was observed in the majority of solvent systems.
Moderate solubility (10>x>100 mg/mL) was observed in dimethylsulfoxide, N-methyl pyrrolidone and deionized water.
XRPD analysis of the solids remaining post-solubility identified phosphate form 1 predominantly.
The poorly crystalline phosphate pattern 2 was observed from ethyl acetate, methylisobutyl ketone and tert-butylmethyl ether.
A mixture of phosphate form 1 and pattern 2 was observed from ethyl formate, isopropyl acetate and methyl ethyl ketone.

TABLE 3
Solvent systems used during the solvent solubility screen.

| | | Volume | Approx. |
|---|---|---|---|
| | | Required | Solubility |
| | Solvent | (mL) | (mg/mL) |
| 1 | 1,4-Dioxane | 2 | <5 |
| 2 | 2-Butanol | 2 | <5 |
| 3 | 2-Ethoxyethanol | 2 | <5 |
| 4 | 2-Methyl tetrahydrofuran | 2 | <5 |
| 5 | 2-Propanol | 2 | <5 |
| 6 | 80% 2-Propanol/20% water (% v/v) | 2 | <5 |
| 7 | 80% Acetone/20% water (% v/v) | 2 | <5 |
| 8 | 80% Acetonitrile/20% water (% v/v) | 2 | <5 |
| 9 | 80% Methanol/20% water (% v/v) | 2 | <5 |
| 10 | Acetone | 2 | <5 |
| 11 | Acetonitrile | 2 | <5 |
| 12 | Anisole | 2 | <5 |
| 13 | Chloroform | 2 | <5 |
| 14 | Dimethylsulfoxide | 0.3 | 33 |
| 15 | Ethanol | 2 | <5 |
| 16 | Ethanol/water Aw 0.4 | 2 | <5 |
| 17 | Ethanol/water Aw 0.5 | 2 | <5 |
| 18 | Ethanol/water Aw 0.7 | 2 | <5 |
| 19 | Ethanol/water Aw 0.8 | 2 | <5 |
| 20 | Ethyl acetate | 2 | <5 |
| 21 | Ethanol/water Aw 0.6 | 2 | <5 |
| 22 | Ethyl formate | 2 | <5 |
| 23 | Ethyleneglycol | 2 | <5 |
| 24 | Isopropyl acetate | 2 | <5 |
| 25 | Methanol | 2 | <5 |
| 26 | Methylethyl ketone | 2 | <5 |
| 27 | Methylisobutyl ketone | 2 | <5 |
| 28 | Nitromethane | 2 | <5 |
| 29 | N-Methyl pyrrolidone | 0.6 | 17 |
| 30 | tert-Butylmethyl ether | 2 | <5 |
| 31 | Tetrahydrofuran | 2 | <5 |
| 32 | Water | 0.6 | 17 |

Phosphate form 1 was produced in the majority of the above samples, with the exception of chloroform (traces of pattern 2 were observed), dimethylsulfoxide (no solid), ethyl acetate (pattern 2), ethyl formate (mix of form 1 and pattern 2), isopropyl acetate (mix of form 1 and pattern 2), methylethyl ketone (mix of form 1 and pattern 2), methylisobutyl ketone (pattern 2), N-methyl pyrolidone (no solid), tert-butylmethylether (pattern 2) and water (no solid).

### Maturation Experiments

The following procedure was used during the polymorph screen maturation experiments:
1 mL aliquots of selected solvent system were added to each sample until about 30 mg of material had dissolved or until 20 mL of solvent was added (maximum volume of vial). The solvents selected are summarised in Table 4.
The samples were then temperature cycled between ambient and 40° C. in 4-hour cycles with agitation for about 48 hours (samples containing water were temperature cycled for about 72 hours). Solvents from which phosphate pattern 2 was likely to be observed from were temperature cycled for 48 hours due to concerns of the stability of the material.
The samples were collected, and observations made.
Observed solids were isolate and analyzed by XRPD.

TABLE 4
Solvents used in maturation experiments.

| | Solvent |
|---|---|
| 1 | 1,4-Dioxane |
| 2 | 2-Butanol |
| 3 | 2-Methyl tetrahydrofuran |
| 4 | 2-Propanol |
| 5 | 80% 2-Propanol/20% water (% v/v) |
| 6 | 80% Acetone/20% water (% v/v) |
| 7 | 80% Acetonitrile/20% water (% v/v) |
| 8 | 80% Methanol/20% water (% v/v) |
| 9 | Acetone |
| 10 | Acetonitrile |
| 11 | Anisole |
| 12 | Chloroform |
| 13 | Ethanol |
| 14 | Ethanol/water Aw 0.4 |
| 15 | Ethanol/water Aw 0.8 |
| 16 | Ethyl acetate |
| 17 | Ethyl formate |
| 18 | Isopropyl acetate |
| 19 | Methanol |
| 20 | Methylethyl ketone |
| 21 | Methylisobutyl ketone |
| 22 | tert-Butylmethyl ether |
| 23 | Tetrahydrofuran |
| 24 | Water |

Phosphate form 1 was observed predominantly in the maturation experiments. A mixture of form 1 and pattern 2 was observed from ethyl formate. Phosphate pattern 2 was observed from tertbutylmethyl ether only.

### Characterization of Phosphate Pattern 2

Phosphate pattern 2 material isolated from tert-butyl methyl ether was collected and dried under vacuum at 40° C. for about 2 hours. The dried material was collected and analyzed by TG/DTA.

The following observations and results were obtained during the characterization of phosphate pattern 2:
TG analysis showed a mass loss of about 0.6% from the onset of heating related to the loss of excess surface solvent.
DT analysis showed a broad melting endotherm from an onset of about 201° C., with a peak at 206° C. The melting endotherm was followed immediately by thermal degradation.
The observed melting point was about 8° C. lower than the melting point of phosphate form 1.
XRPD analysis indicated that phosphate pattern 2 samples had all converted to form 1 upon storage at 40° C./75% RH.

The XRPD multi-well plate containing the phosphate pattern 2 material observed in the solvent solubility screen was collected and stored in a stability chamber at 40° C./75% RH for about 16 hours. The samples were analyzed by XRPD.

The phosphate pattern 2 was determined to have lower melting point than form 1, indicating the material is less stable. Upon storage at 40° C./75% RH phosphate pattern 2 material completely converted to form 1, confirming pattern 2 to be a metastable form of the phosphate salt.

### Example 5—Preparation of L-Tartrate Salt of 1-methyl-1,4,5,10-tetrahydropyrazolo[3,4-b][1,5]benzodiazepine

The 1-methyl-1,4,5,10-tetrahydropyrazolo[3,4-b][1,5]benzodiazepine L-tartrate salt was prepared using the following procedure:
Approximately 500 mg of 1-methyl-1,4,5,10-tetrahydropyrazolo[3,4-b][1,5]benzodiazepine freebase was weighed into a 20 mL scintillation vial.
5 mL of ethanol was added to the material to form a mobile slurry.
2.62 ml (1.05 equivalents) of 1M L-tartaric acid stock solution (prepared in THF) was added to the material slurry. A small amount of beige solid suspended in a dark pink solution was observed.
The slurry was then temperature cycled between ambient and 40° C. in 4-hours cycles with stirring for about 24 hours. After about 2 hours stirring at 40° C. a thick off-white slurry was observed.
The sample was collected after about 24 hours, and a small amount of material was isolated using a plastic pipette and analyzed by XRPD whilst damp.
The bulk material was filtered using a Buchner funnel and dried on the filter bed for about 1 hour. The material filtered slowly and a thick, agglomerated, off-white powder was observed.
The bulk material was dried under vacuum at 40° C. for about 16 hours.
The dried material was analyzed by XRPD.

The dried material was fully characterised by TG/DTA, DSC, DVS with post-DVS, XRPD, HPLC, ¹H NMR and LC-MS, as per the methods detailed above.

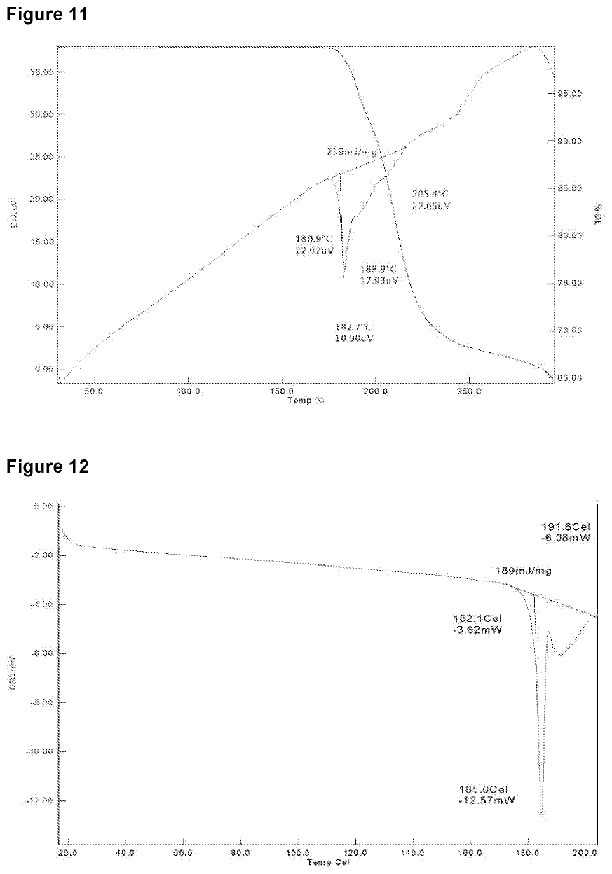

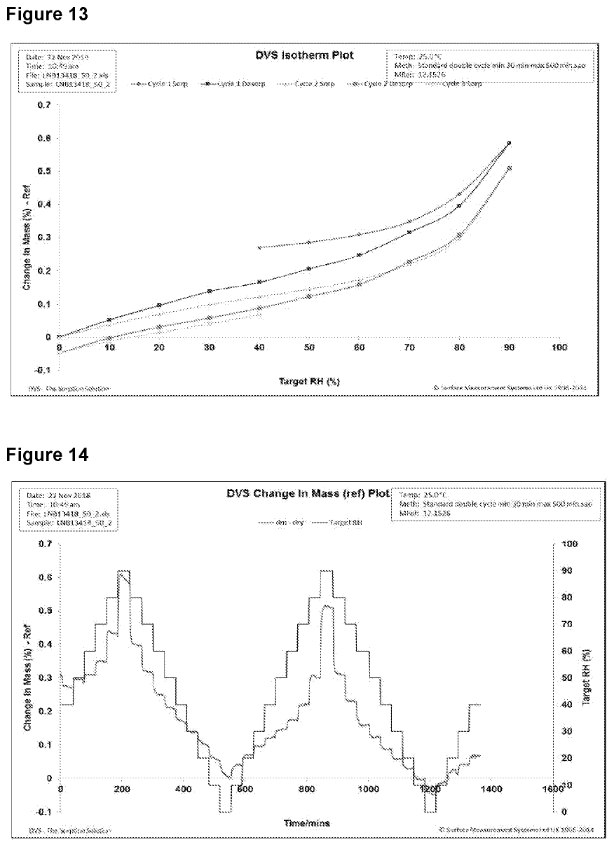

The isolated material appeared highly crystalline by XRPD (FIG. 10).
The PLM micrographs show small, birefringent agglomerates of particles with no defined morphology (not shown) possibly due to the use of stirrer bars in the scale up process.
TG analysis showed no mass loss until thermal degradation above about 200° C.
DT analysis showed a large endothermic melting transition with thermal degradation from an onset of about 181° C. with a peak at 183° C. (FIG. 11).
DSC analysis showed a large endothermic melting transition from an onset of about 182° C. with a peak at 185° C. (FIG. 12). No thermal events were observed during the cooling and second heat steps. DSC data corresponds with the TG/DTA data.
The material appeared slightly hygroscopic by DVS with a mass increase of about 0.6 wt. % at 90% RH. No changes in form were observed in the DVS kinetic plot. The hysteresis observed in the isotherm plot is indicative of crystallization of amorphous content during the experiment (FIG. 13 and FIG. 14).
XRPD analysis of the post-DVS material showed no changes in form.
It was determined by ¹H NMR that minor impurities were present in the sample of L-tartrate salt which were also identified in the input L-tartaric acid.
HPLC analysis confirmed a high purity (by area %) of 99.0%.

The L-tartrate form 1 salt was successfully prepared by the above process. The isolated material appeared highly crystalline by XRPD and PLM. The observed crystals showed no defined morphology by PLM which may be due to the use of a stir bar during preparation. Agglomeration of particles was observed in the PLM analysis. TG analysis confirmed the material was anhydrous. DT and DSC analysis identified a melting point of about 181° C., consistent with the primary screen data. The material appeared slightly hygroscopic by DVS with a mass increase of about 0.6% at 90% RH. No evidence of form changes or hydrate formation was observed by XRPD post-DVS analysis. The collected ¹H NMR spectrum showed the expected connectivity with the structure provided. Approximately 1 equivalent of L-tartaric acid was observed in the ¹H NMR spectrum. Small impurities were observed in the ¹H NMR spectrum which were later identified in the L-tartaric acid input material. HPLC analysis confirmed a high purity of 99.0% (by area %), showing a small uplift in purity from the freebase input material.

### Example 6-1 Week Stability Studies

One-week stability studies were conducted on the freebase (example 1), phosphate form 1 (example 3) and L-tartrate form 1 (example 5) salts using the following procedures:
Approximately 20 mg of each form was weighed into 3×2 mL glass vials.
One vial for each form was stored under the following conditions for 1-week:
   1. Ambient temperature, light and humidity (unsealed vial)
   2. 40° C./75% RH (unsealed vial)
   3. 80° C. (sealed vial)
The samples were collected after 1-week and observations were made.
The solids were analyzed by XRPD and HPLC.

During the 1-week stability studies, no changes in form were identified in the phosphate form 1 and L-tartrate form 1 salts by XRPD. HPLC analysis did not identify any significant changes in purity in the salt samples.

The freebase samples stored at 40° C./75% RH and 80° C. showed no changes in form or purity. An additional peak was observed in the XRPD diffractogram of the freebase sample stored under ambient conditions, indicating a potential change in form or degradation. An additional XRPD pattern collected after 2 weeks showed further changes in diffraction pattern. HPLC analysis did not show any significant changes in purity. The data indicates that the freebase may be unstable under ambient light, but otherwise stable under thermal conditions.

### Example 7—Salt Disproportionation and Hydration Studies

The following experiments were conducted to assess the likelihood of salt disproportionation of the phosphate form 1 and L-tartrate form 1 salts in deionised water and the potential for hydrate formation using solvent/water mixtures with various water activities.

The following procedure was used during the salt disproportionation studies:
Approximately 20 mg of phosphate form 1 (Example 3) and L-tartrate form 1 (Example 5) were weighed into 2 mL glass vials.
Deionized water was added to each sample in 100 μL aliquots until a mobile slurry was observed.
The slurries were agitated at ambient temperature for about 24 hours.
The slurries were collected and filtered via centrifugation.
The isolated solids were analyzed by XRPD.
The pH of the mother liquors were recorded.

The following procedure was used during the hydration studies:
Approximately 20 mg of phosphate form 1 (Example 3) and L-tartrate form 1 (Example 5) were weighed into 2 mL glass vials.
Selected methanol/deionized water mixtures (see Table 5) were added to each sample in 100 μL aliquots until a mobile slurry was observed.
The slurries were agitated at ambient temperature for about 24 hours.
The slurries were collected and filtered via centrifugation.
The isolated solids were analyzed by XRPD.
The pH of the mother liquors were recorded.

TABLE 5
Solvent/water mixtures used in the salt
disproportionation and hydration studies.

| | | | Approximate |
|---|---|---|---|
| Sample | Salt | Solvent System | Water Activity |
| 1 | Phosphate Form 1 | 95% MeOH:5% Water | 0.2 |
| 2 | Phosphate Form 1 | 70% MeOH:30% Water | 0.6 |
| 3 | Phosphate Form 1 | 35% MeOH:65% Water | 0.8 |
| 4 | Tartrate Form 1 | 95% MeOH:5% Water | 0.2 |
| 5 | Tartrate Form 1 | 70% MeOH:30% Water | 0.6 |
| 6 | Tartrate Form 1 | 35% MeOH:65% Water | 0.8 |

The following results were obtained during the salt disproportionation studies of phosphate form 1 (example 3) and L-tartrate form 1 (example 5) salts:
Salt disproportionation was not observed in the salt samples.
No change in XRPD pattern was observed in the phosphate pattern 1 material slurried in water.
No change in XRPD pattern was observed in the L-tartrate pattern 1 material slurried in water.

The following results were obtained during the hydration studies of phosphate form 1 (example 3) and L-tartrate form 1 (example 5) salts:
Hydrate formation was not observed in the salt samples in the solvent/water mixtures.
No change in XRPD pattern was observed in the KNX-100 phosphate pattern 1 samples.
No change in XRPD pattern was observed in the KNX-100 L-tartrate pattern 1 samples.

No evidence of hydrate formation or salt disproportionation was observed in the phosphate form 1 and L-tartrate salts.

### Example 8—Thermodynamic Solubility Assessment

The thermodynamic solubility of freebase (example 1), phosphate form 1 (example 3) and L-tartrate form 1 (example 5) were assessed in phosphate buffered saline (PBS) buffer at pH 7.4 according to the following procedure:
Approximately 30 mg of each form was weighed into 2 mL glass vials.
100 μL of buffer solution was added to each vial until partial dissolution was observed.
The observed slurries were agitated at ambient temperature for about 24 hours.
The samples were collected, and observations were recorded.
The samples were filtered via centrifugation.
The pH of the mother liquors were recorded.
The concentration of the mother liquors were recorded by HPLC.

The following results (summarised in Table 6) were obtained during the thermodynamic solubility assessment of freebase (example 1), phosphate form 1 (example 3) and L-tartrate form 1 (example 5):
HPLC analysis identified a solubility of 0.3 mg/mL for the freebase in PBS buffer at pH 7.4.
HPLC analysis identified a solubility of 7.4 mg/mL for the phosphate form 1 salt in PBS buffer at pH 7.4.
HPLC analysis identified a solubility of 6.3 mg/mL for the L-tartrate form 1 salt in PBS buffer at pH 7.4.

TABLE 6
Solubility of solid forms of the compound of the invention

| | Volume of | Initial | Observations | pH | Solubility |
|---|---|---|---|---|---|
| Form | Buffer(μL) | Observations | (t = 24 h) | (t = 24 h) | (mg/mL) |
| Freebase | 2000 | Beige Slurry | Beige Slurry | 7.32 | 0.3 |
| Phosphate Form 1 | 2000 | Pink Slurry | Pink Slurry | 3.48 | 7.4 |
| Tartrate Form 1 | 1000 | Pale pink | Pale pink | 3.36 | 6.3 |
| | | slurry | slurry/pink | | |
| | | | crystals | | |

During the thermodynamic solubility screen, the KNX-100 freebase was found to have the lowest solubility in PBS buffer at pH 7.4, with a solubility of 0.3 mg/mL. Higher solubilities were identified in the salt forms. KNX-100 L-tartrate form 1 had a solubility of 6.3 mg/mL and the KNX-100 phosphate pattern 1 was found to have the highest solubility of 7.4 mg/mL.

### Example 9—Pharmacokinetic Properties of the Phosphate Form 1 and the Dihydrochiride Salt of the Compound of the Invention

This Example describes pharmacokinetic experiments in male Sprague Dawley rats. The Example show that oral administration of the compound of the invention (CMPD1) as phosphate form 1 leads to the same exposure profile as the compound of the invention dosed in dihydrochloride salt form, whether the drug is administered using a saline or methocel vehicle.

### Method

N=3 rats were run in each of the four conditions:
(1) Di-HCL salt in saline
(2) Di-HCL salt in methocel
(3) Phosphate form 1 in saline
(4) Phosphate form 1 in methocel

On the day of dosing the solid compounds were dissolved in either Saline (0.9%) or 0.5% hydroxypropyl methylcellulose (Methocel E3 Premium LV) in Milli-Q water. The formulations were then thoroughly vortexed to produce colourless solutions.

Overnight-fasted rats with adlibitum access to water were administered their dose of the various forms of the compound of the invention via oral gavage (PO) at a dose volume of 3 ml/kg and a dose of 5 mg/kg freebase equivalent. Food access was re-instated 4 hours (h) post-dose. Samples of arterial blood were collected up to 24 h post-dose. After collection, samples were centrifuged, plasma was removed and stored frozen at −80° C. before being analysed by LC-MS. Urine samples were collected at pre, 0-4 h, 4-7 h and 7-24 h post-dose and were analysed by LC-MS following extraction.

### Results

No adverse reactions or compound-related side effects were observed in any rats during the 24 h sampling period after dosing.

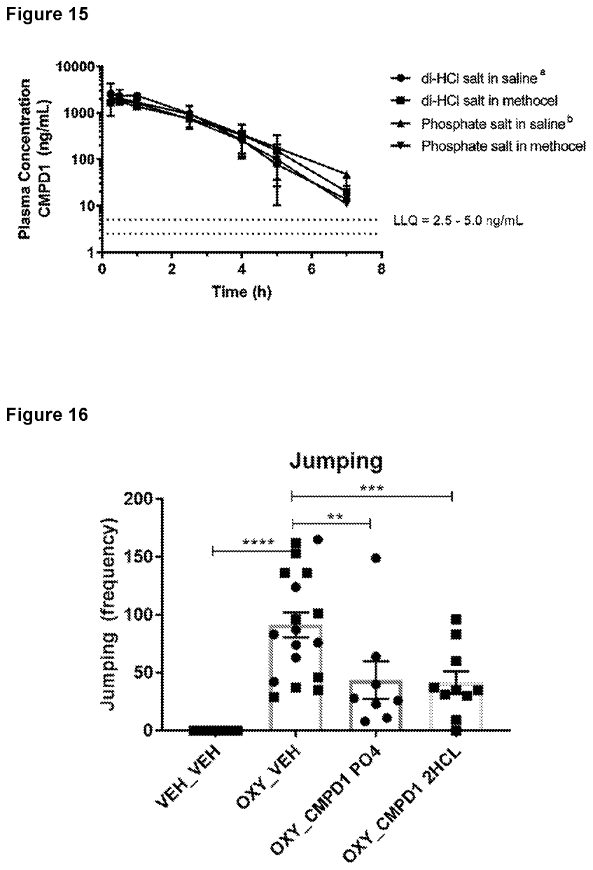

The mean plasma concentration of the compound of the invention versus time profiles of following oral administration of the di-hydrochloride salt and phosphate form 1 in each formulation are shown in FIG. 15, and the mean exposure parameters for the compound of the invention are summarised in Table 7.

TABLE 7
Mean plasma exposure parameters of compound of the invention in male Sprague
Dawley rats following oral administration of compound of the invention
as its di-hydrochloride and phosphate salts (form 1) in saline and methocel
formulations. All doses and concentrations are expressed as the freebase
equivalents. Data are shown as mean ± SD, n = 3 for all groups

| | CMPD1, 5 mg/kg Target Dose | | | |
|---|---|---|---|---|
| | Di-hydrochloride salt | | Phosphate salt | |
| Parameter | Saline^{a} | Methocel | Saline | Methocel |
| Calculated dose (mg/kg) | 4.8 ± 0.03 | 4.5 ± 0.02 | 4.9 ± 0.02 | 4.9 ± 0.01 |
| Apparent t_{1/2} (h) | 0.69 ± 0.10 | 0.68 ± 0.2 | 0.81 ± 0.2 | 0.60 ± 0.02 |
| Plasma Cₘₐₓ (μg/mL) | 2.93 ± 1.36 | 1.80 ± 0.11 | 2.09 ± 0.14 | 2.01 ± 0.25 |
| Tₘₐₓ (h) | 0.75 ± 0.4 | 0.33 ± 0.1 | 0.50 ± 0.4 | 0.25 ± 0 |
| Plasma AUC_{0-inf} (μg*h/mL) | 5.46 ± 0.54 | 4.10 ± 0.52 | 4.95 ± 1.75 | 4.16 ± 0.88 |
| BA % | 124 ± 13 | 98.9 ± 13^{b} | 111 ± 40^{b} | 93.2 ± 20^{b} |
| % Dose in urine^{c} | 0.184 ± 0.059 | 0.224 ± 0.14 | 0.395^{d} | 0.222 ± 0.093 |

Dose-Normalised Parameters

| | | | | |
|---|---|---|---|---|
| Plasma Cₘₐₓ/Dose | 0.61 ± 0.29 | 0.40 ± 0.02 | 0.43 ± 0.03 | 0.41 ± 0.05 |
| (μg/mL)/(mg/kg) | | | | |
| Plasma AUC_{0-inf}/Dose | 1.14 ± 0.12 | 0.91 ± 0.12 | 1.02 ± 0.36 | 0.85 ± 0.18 |
| (μg*h/mL)/(mg/kg) | | | | |

The plasma concentration versus time profiles of the compound of the invention were closely comparable for all four treatment groups. This was reflected in the similar dose-normalised Cmax and AUC_{0-inf} values, suggesting that neither the salt form nor formulation vehicle had any substantial impact on the exposure of the subject to the compound of the invention.

### Example 10—Biological Efficacy of the Phosphate Form 1 Compared with the Dihydrochloride Salt of the Compound of the Invention

This Example describes experiments in a C57BL/6 mouse model of opioid withdrawal (naloxone precipitated withdrawal following oxycodone administration) and the potential of the compound of the invention in two different salt forms, administered at the same freebase equivalent dose, to treat withdrawal symptoms. This experiment confirms that a substantially similar biological activity is achieved for phosphate form 1 as for previously described forms of the compound of the invention.

### Experiment 10.1: Assessing the Effects of the Compound of the Invention as a Dihydrochloride Salt (CMPD1-2HCL) and as Phosphate Form 1 (CMPD1-PO4) on Naloxone Precipitated Oxycodone Withdrawal

Two cohorts of adult male C57BL/6 mice were run assessing the effects of 7.3 mg/kg freebase equivalent (FBE) IP of the compound of the invention in the dihydrochloride salt form (CMPD1-2HCL) and the phosphate form 1 (CMPD1-PO4) on naloxone precipitated withdrawal-induced jumping.

The first cohort of mice (N=30) were split into the following conditions:
(1) Vehicle, 0 mg/kg (n=10);
(2) oxycodone, 0 mg/kg compound of the invention FBE in 2HCL salt form (n=10);
(3) oxycodone, 7.3 mg/kg compound of the invention FBE in 2HCL salt form (n=10).

The second cohort of mice (N=24) were split into the following conditions:
(1) Vehicle, 0 mg/kg (n=8);
(2) oxycodone, 0 mg/kg compound of the invention FBE in PO4 salt form (n=8);
(3) oxycodone, 7.3 mg/kg compound of the invention FBE in PO4 salt form (n=8).

Mice in the oxycodone conditions received i.p. injections of oxycodone for 5 days according to the schedule and doses set out in Table 8. The morning and afternoon doses were separated by 7 h. Mice in the vehicle condition received injections of vehicle saline instead of oxycodone. One-hour-and-forty-five minutes after the morning injection on day 5, mice were administered their i.p. dose of the compound of the invention. Fifteen minutes later they received an i.p. injection of 10 mg/kg naloxone (oxycodone groups) or saline (vehicle group), and proceeded immediately to testing.

TABLE 8
Oxycodone dosing schedule for mice in the oxycodone conditions.

| Day: | 1 | | 2 | | 3 | | 4 | | 5 | |
|---|---|---|---|---|---|---|---|---|---|---|
| Time of day: | AM | PM | AM | PM | AM | PM | AM | PM | AM | PM |
| Oxycodone dose (mg/kg i.p.) | 9 | 9 | 17.8 | 17.8 | 23.7 | 23.7 | 23.7 | 23.7 | 23.7 | — |

Testing involved placing mice individually into a 20 (I)×20 (w)×30 (h) cm arena for 30 min. Sessions were captured via a side view high speed (120 fps), high resolution (4K) camera. Number of jumps were scored from the videos by an experienced experimenter blind to conditions.

Data were analysed by SPSS using oneway ANOVA and planned contrasts.

### Jumping

Data for jumping are shown in FIG. 16. Data from Cohort 1 mice are shown with square symbols, data from Cohort 2 mice are shown with circle symbols.

The overall ANOVA assessing jumping was significant [F3,50=21.52, p<0.0001]. Planned contrasts revealed mice undergoing oxycodone withdrawal jumped significantly more times during the test session [VEH_VEH vs OXY_VEH, p<0.0001].

7.3 mg/kg FBE compound of the invention in both the phosphate (CMPD1-PO4) and 2HCl (CMPD1-2HCL) salt form significantly inhibited oxycodone withdrawal-induced jumping at all doses tested [OXY_VEH vs: OXY_CMPD1-PO4, p<0.01; OXY_CMPD1-2HCL, p<0.001]. Moreover, the results following dosing FBE of the two salt forms did not differ significantly from each other in withdrawal-induced jumping [OXY_CMPD1-PO4 vs OXY_2HCL, p=0.901] (FIG. 16).

1. A phosphoric acid addition salt of 1-methyl-1,4,5,10-tetrahydropyrazolo[3,4-b][1,5]benzodiazepine.

2. The phosphoric acid addition salt of claim 1 in a crystalline form characterised by strong peaks in an X-ray diffraction (XRD) pattern at about 12° and about 18° 2θ.

3. The phosphoric acid addition salt of claim 1 or 2 characterised by peaks in the XRD pattern at about 12°, 14°, 17.5°, 18°, 19°, 20°, 20.8°, 22.5°, 24°, 24.8°, 26°, 26.5° and 27.8° 2θ.

4. The phosphoric acid addition salt of any one of claims 1-3 having a melting point of about 200° C.

5. The phosphoric acid addition salt of any one of claims 1-4 as an anhydrous crystal.

6. An L-tartaric acid addition salt of 1-methyl-1,4,5,10-tetrahydropyrazolo[3,4-b][1,5]benzodiazepine.

7. The L-tartaric acid addition salt of claim 6 in crystalline form.

8. The L-tartaric acid addition salt of claim 7 as an anhydrous crystal.

9. A crystal of 1-methyl-1,4,5,10-tetrahydropyrazolo[3,4-b][1,5]benzodiazepine.

10. The crystal of claim 9 as an anhydrous crystal.

11. A process for preparing the phosphoric acid addition salt of any one of claims 1-5, comprising
preparing a solution comprising 1-methyl-1,4,5,10-tetrahydropyrazolo[3,4-b][1,5]benzodiazepine freebase, a solvent, and phosphoric acid; and
separating excess solvent and phosphoric acid to provide the phosphoric acid addition salt.

12. A process for preparing the phosphoric acid addition salt of any one of claims 1-5, comprising reacting N-(1-methyl-1H-pyrazol-5-yl)-benzene-1,2-diamine with formaldehyde in the presence of phosphoric acid in a solvent.

13. The process of claim 11 or 12, wherein the solvent is selected from 1,4-dioxane, 2-butanol, 2-ethoxyethanol, 2-methyl tetrahydrofuran, 2-propanol, acetone, acetonitrile, methanol, anisole, ethanol, tetrahydrofuran, ethyleneglycol and water or a combination thereof.

14. The process of any one of claims 11 to 13, wherein the solvent does not comprise ethyl acetate, methylisobutyl ketone, tert-butylmethyl ether, ethyl formate, isopropyl acetate and methyl ethyl ketone or a combination thereof.

15. A process for preparing the L-tartaric acid addition salt of any one of claims 6 to 8, comprising:
preparing a solution comprising 1-methyl-1,4,5,10-tetrahydropyrazolo[3,4-b][1,5]benzodiazepine freebase, a solvent, and L-tartaric acid; and
separating excess solvent and L-tartaric acid from the L-tartaric acid addition salt.

16. A process for preparing a crystalline form of 1-methyl-1,4,5,10-tetrahydropyrazolo[3,4-b][1,5]benzodiazepine freebase, comprising:
preparing a solution of a hydrochloride salt of 1-methyl-1,4,5,10-tetrahydropyrazolo[3,4-b][1,5]benzodiazepine and an aqueous saturated bicarbonate solution; and
contacting the solution with an organic solvent to extract 1-methyl-1,4,5,10-tetrahydropyrazolo[3,4-b][1,5]benzodiazepine freebase from the solution.

17. A medicament comprising the salt of any one of claims 1 to 8 or the crystal of claim 9 or 10.

18. A pharmaceutical composition comprising the salt of any one of claims 1 to 8 or the crystal of claim 8 or 9.

19. A method of:
treating or preventing antisocial behaviour in a subject, and/or
providing acute and long-term regulation of social behavior in a subject, and/or
treating or preventing a substance abuse disorder in a subject, and/or
treating or preventing a social dysfunction in a subject, and/or
treating or preventing a psychiatric disorder in a subject as part of a therapy for a psychiatric disorder that features social dysfunction as a primary or secondary feature; and/or
causing weight loss in a subject; and/or
managing weight in a subject; and/or
suppressing an appetite for food in a subject; and/or
reducing the consumption of food by a subject; and/or
treating or preventing opioid withdrawal and/or a symptom thereof in a subject;
the method comprising administering to the subject an effective amount of the salt of any one of claims 1 to 8, the crystal of any one of claims 9 or 10, the medicament of claim 17, or the pharmaceutical composition of claim 18.

20. A method of treating a subject suffering from or at risk of developing a substance abuse disorder, or a subject recovering from a substance abuse disorder and seeking to maintain ongoing abstinence of the substance, comprising administering to the subject an effective amount of the salt of any one of claims 1 to 8, the crystal of any one of claims 9 or 10, the medicament of claim 17, or the pharmaceutical composition of claim 18, to thereby treat or prevent the substance abuse disorder.

21. Use of the salt of any one of claims 1 to 8 or the crystal of claim 9 or 10 in the manufacture of a medicament for:
treating or preventing antisocial behaviour in a subject, and/or
providing acute and long-term regulation of social behavior in a subject, and/or
treating or preventing a substance abuse disorder in a subject, and/or
treating or preventing a social dysfunction in a subject, and/or
treating or preventing a psychiatric disorder in a subject as part of a therapy for a psychiatric disorder that features social dysfunction as a primary or secondary feature; and/or
causing weight loss in a subject; and/or
managing weight in a subject; and/or
suppressing an appetite for food in a subject; and/or
reducing the consumption of food by a subject; and/or
treating or preventing opioid withdrawal and/or a symptom thereof in a subject.
